(19) United States
(12) Patent Application Publication
(10) Pub. No.: US 2026/0049361 A1
(43) Pub. Date: Feb. 19, 2026

(54) LOWER RESPIRATORY TRACT INFECTIONS

(71) Applicants: CZ Biohub SF, LLC, San Francisco, CA (US); The Regents of the University of California, Oakland, CA (US); Chan Zuckerberg Initiative Foundation, Redwood City, CA (US); The Regents of the University of Colorado, a body corporate, Denver, CO (US)

(72) Inventors: Charles R. Langelier, Pacifica, CA (US); Peter Mourani, Little Rock, AR (US); John A. Kamm, San Francisco, CA (US); Eran Mick, San Francisco, CA (US); Alexandra Tsitsiklis, Berkeley, CA (US); Katrina Kalantar, Redwood City, CA (US); Joseph L. DeRisi, Moraga, CA (US)

(21) Appl. No.: 19/120,435

(22) PCT Filed: Oct. 26, 2023
(86) PCT No.: PCT/US2023/077971

§ 371 Date: Apr. 11, 2025

Related U.S. Application Data
(60) Provisional application No. 63/381,156, filed on Oct. 27, 2022.

(51) Int. Cl. C12Q 1/689; C12Q 1/6809; C12Q 1/6844; C12Q 1/6869; C12Q 1/70; G16B 25/10; G16B 40/20

(52) U.S. Cl. CPC C12Q 1/689; C12Q 1/6809; C12Q 1/6844; C12Q 1/6869; C12Q 1/70; G16B 25/10; G16B 40/20; C12Q 2600/118; C12Q 2600/166

(57) ABSTRACT

Various embodiments are directed to analyzing host gene expression levels and microbial diversity in a biological sample, to determine a likelihood of lower-respiratory tract infection (LRTI) in subjects. Embodiments can include determining a probability value of a subject having LRTI based on differential gene expression of the subject and reference levels of control subjects. Embodiments can also include determining the likelihood of LRTI in subjects based on amicrobial diversity index or abundance levels of microbes that are considered as potential pathogens. Embodiments can also include applying an integrated classifier to gene expression levels, virus abundance levels, and microbial diversity to determine the likelihood of LRTI in subjects.

### CROSS-REFERENCES TO RELATED APPLICATION

This application is a PCT of and claims the benefit of U.S. Provisional Patent Application No. 63/381,156, entitled “Diagnosis of Lower Respiratory Tract Infections,” filed on Oct. 27, 2022, which is herein incorporated by reference in its entirety for all purposes.

### BACKGROUND

Lower Respiratory Tract Infections (LRTI) are infections that affect the airways (below the level of the larynx), including the trachea and the alveoli. For example, LRTIs can be pneumonia which corresponds to infections in air sacs of the lungs. LRTIs can be caused by various types of pathogens, including viral pathogens such as Respiratory Syncytial Virus and RSV and bacterial pathogens such as Streptococcus pneumoniae, Heamophilus influenzae, and Moraxella catharralis. The ability to accurately distinguish LRTI from other inflammatory lung diseases and rapidly detect the etiologic pathogens is thus needed for implementing effective, targeted therapies.

However, existing microbiologic diagnostic techniques are limited in terms of low sensitivity, low turnaround time, and narrow spectrum of pathogen targets. As such, LRTI treatment in many cases is empirical, which leads to antimicrobial overuse, selection for resistant pathogens, and occurrence of adverse events in a significant fraction of patients. For example, for a patient showing symptoms associated with LRTI, existing diagnostic tests for detecting LRTI-causing pathogens depend on bacterial culture and take several days. To treat the infection, the patient is initially treated empirically with broad spectrum antibiotics until the diagnostic tests provide the results of whether the LRTI was caused by bacterial or viral pathogens. But the antibiotic treatment may end up being inappropriate (e.g., the patient has a viral infection) and may cause adverse events for the patient. In intensive care units, LRTI diagnosis can be particularly complex due to non-infectious, systemic inflammatory conditions that may be clinically indistinguishable from LRTI. Due to such limitations and potential for misdiagnosis, LRTIs cause more deaths each year than any other type of infection. Further, LRTIs burden disproportionately affect children.

Recently, profiling host gene expression from blood samples has shown promise as an innovative modality for diagnosing LRTI in hospitalized patients. Such approach, while a significant step forward, remains unproven in critically ill pediatric population. Further, because assessment is typically carried out in peripheral blood versus at the site of active infection in the respiratory tract, gene expression alone is unable to identify the relevant LRTI pathogens, which is needed for optimal antimicrobial therapy.

Metagenomic next generation sequencing (mNGS) of the lower airway (tracheal aspirate, TA) has been used to identify host gene expression signatures of LRTI and detect pathogens in a prospective cohort of mechanically ventilated adults. However, it has not been demonstrated whether mNGS alone can be successfully applied in different types of patient populations (e.g., pediatric patients) due to age-related differences in LRTI epidemiology, rates of asymptomatic pathogen carriage, and immune responses to infection.

Accordingly, there is a need to build a better diagnostic test for infections of all different types, as well as certain populations that are vulnerable to infections including LRTIs. For example, the diagnostic test needs to be designed for critically ill children who are admitted to the hospital with severe respiratory infections.

### SUMMARY

Various embodiments are directed to applications of the analysis of biological samples (e.g., tracheal aspirate samples) to determine a likelihood of lower-respiratory tract infection in a subject. For example, tracheal aspirate samples of subjects with acute respiratory failure can be used to profile host gene expression and respiratory microbiota. A host classifier can be used to process host gene expression levels to determine whether a subject has an increased likelihood of LRTI. RNA of the subject in the biological sample from each member of a gene panel can be detected, in which the gene panel comprises at least two members selected from a group consisting of GNLY, PSMB8, FFAR3, SLC38A2, ISG15, IRF1, KIAA1841, AC090425.2, AKR1C3, CXCL5, SESN1, PCOLCE2, RBP4, TAP1, EPSTI1, and FABP4. From the detected RNA, a quantity of differential gene expression for each member of the gene panel compared to reference levels of RNA in control subjects can be determined. Then, a probability value can be determined based on the respective quantities of differential gene expression, at which it can be determined that the subject has an increased likelihood of lower-respiratory tract infection based on the probability value exceeding a threshold value, e.g., relative to subjects with the probability value being below the threshold value.

In some instances, the host classifier can be trained on training data that include: (i) patients with a diagnosis of LRTI supported by microbiologic findings (n=117); and (ii) patients with respiratory failure due to non-infectious causes (n=50). The host classifier resulted in very high accuracy in diagnosing LRTI in subjects, achieving a median AUC of 0.967 by 5-fold cross-validation.

To further enhance the diagnostic accuracy, an integrated meta-classifier can be implemented. In particular, the integrated meta-classifier can be used to determine an increased likelihood of LRTI in subjects based on: (i) the LRTI probability value generated by the host classifier; (ii) an abundance of respiratory viruses in the biological sample; and (iii) a relative dominance of bacteria/fungi deemed potential pathogens according to a rules-based model (diversity model). The integrated classifier achieved a median AUC of 0.986 by 5-fold cross-validation. When applied to patients with suspected or indeterminate LRTI status (n=94), the integrated classifier indicated LRTI in 52% of cases and identified likely pathogens in 98% of those. Thus, the integrated classifier demonstrates the feasibility of accurate LRTI diagnosis and pathogen identification in critically ill subjects using lower airway metagenomics.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings. Before the disclosure is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### BRIEF DESCRIPTION OF THE DRAWINGS

FIG. 1 shows a schematic diagram that illustrates a process for determining a likelihood of LRTIs in subjects, according to some embodiments.

FIG. 2 shows a schematic diagram that illustrates a screening process 200 for selecting a cohort for determining a likelihood of LRTIs in subjects, according to some embodiments.

FIG. 3 shows an example set of graphs 300 illustrating a correlation of gene expression and classification of LRTI status, according to some embodiments.

FIG. 4 is a flowchart for a method 400 for determining a likelihood of LRTI in a subject based on gene expression levels, according to some embodiments.

FIG. 5 shows a set of graphs that identify classification accuracy of the trained classifier, according to some embodiments.

FIG. 6 shows a heatmap showing the normalized expression across samples (columns) of the 14 final classifier genes (rows) selected when training on the complete Definite and No Evidence dataset.

FIG. 7 shows an example set of graphs 700 that identify expression of the top eight host classifier genes by coefficient in LRTI^{definite} (red) and No-LRTI (blue) in subjects of different ages, according to some embodiments.

FIG. 8 is a flowchart for a method 800 for using machine-learning techniques to determine a likelihood of LRTI in a subject, according to some embodiments.

FIG. 9 shows a set of graphs that identify abundance levels of viruses after background filtering, according to some embodiments.

FIG. 10 shows a set of graphs that identify a comparison between using mNGS and PCR for detecting viruses in subjects diagnosed with LRTI, according to some embodiments.

FIG. 11 illustrates example processes for determining potential pathogens that contribute to LRTI in subjects, according to some embodiments.

FIG. 12 shows a set of graphs that identify different characteristics of microbes detected by using the diversity model, according to some embodiments.

FIG. 13 shows a set of graphs that identify a comparison between using mNGS and culture tests for detecting bacterial pathogens in subjects diagnosed with LRTI, according to some embodiments.

FIG. 14 shows a set of boxplots 1400 that show a correlation between microbial diversity and occurrence of LRTIs in subjects, according to some embodiments.

FIG. 15 shows a set of graphs 1500 that show a correlation between gene expression in subjects with underlying type of infections, according to some embodiments.

FIG. 16 shows a set of graphs 1600 that identify the difference of gene expression levels between co-infection samples and virus-only infection samples, according to some embodiments.

FIG. 17 is a flowchart for a method 1700 for using pathogen abundance levels to determine a likelihood of LRTI in a subject, according to some embodiments.

FIG. 18 shows a schematic diagram 1800 for using an integrated classifier to determine a likelihood of LRTI in subjects, according to some embodiments.

FIG. 19 shows a scatterplot 1900 of the host LRTI probability (x-axis) and the sum of the log₁₀-transformed microbial features (y-axis) in the Definite and No Evidence patients.

FIG. 20 shows a set of graphs 2000 that identify evaluation results of the integrated classifier, according to some embodiments.

FIG. 21 shows comparison data 2100 between the probability of LRTI derived from the host classifier and the integrated classifier for Definite (left panel) 2102 and No Evidence (right panel) 2104 subjects.

FIG. 22 shows evaluation results 2200 of the integrated classifier on subject suspected of LRTI, according to some embodiments.

FIG. 23 also shows a visual summary 2300 incorporating all three inputs of the integrated classifier and its output LRTI probability for Suspected and Indeterminate cases.

FIG. 24 is a flowchart for a method 2400 for using an integrated classifier to determine a likelihood of LRTI in a subject, according to some embodiments.

FIG. 25 illustrates a measurement system 2500 according to an embodiment of the present disclosure.

FIG. 26 shows a block diagram of an example computer system usable with systems and methods according to embodiments of the present disclosure.

### TERMS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “an agent” includes reference to one or more agents known to those skilled in the art, and so forth.

A “biological sample” or “sample,” as used herein, generally refers to a substance obtained from a subject, e.g., a human subject. A biological sample contains analytes for example those described herein, i.e., nucleic acids, such as human RNA expressed by cells of the subject and potentially microbial RNA (e.g., virus, bacteria, fungi) that may cause LRTI. In some embodiments, a biological sample is a sample comprising cells from the nose, mouth, throat or lower respiratory tract of the subject. A sample from the nose or mouth may be collected, for example, by a buccal swab, nasal swab, nasopharyngeal swab, nasopharyngeal wash or aspirate, mid-turbinate nasal swab, oropharyngeal swab, or saliva specimen. In some embodiments, the biological sample is a sample comprising fluid from the lungs, such as a broncho-alveolar lavage, or an endotracheal aspirate. In one embodiment, the biological sample is a sample comprising cells from the nose and is collected with a nasal swab. In one embodiment, the biological sample is a sample comprising cells from the nose and is collected with a nasopharyngeal swab. In one embodiment, the biological sample is a sample comprising cells from the throat and is collected with an oropharyngeal swab. In some embodiments, solid tissues, for example lung tissues, may be used as biological samples. Additional biological samples include serum, plasma, or blood. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

The terms “determining,” “assessing,” “assaying,” “measuring” and “detecting” with respect to assessing LRTI-associated patient RNA profiles refer to quantitative determinations.

As used herein, the term “differentially expressed” refers to differences in the expression level or abundance (i.e., in the quantity and/or the frequency) of a gene marker (e.g., RNA) present in a sample taken from patients having LRTI as compared to reference levels in control subjects, e.g., subjects having a clear non-infectious cause of acute respiratory failure and no clinical or microbiologic suspicion of LRTI. For example, the transcript or RNA levels of a gene marker may be present at an elevated level or at a decreased level in samples of patients with LRTI compared to the reference levels.

As used herein, the terms “cutoff” and “threshold” refer to predetermined numbers used in an operation. A threshold value may be a value above or below which a particular classification applies. Either of these terms can be used in either of these contexts. A cutoff or threshold may be “a reference value” or derived from a reference value that is representative of a particular classification or discriminates between two or more classifications. Such a reference value can be determined in various ways, as will be appreciated by the skilled person. For example, metrics can be determined for two different groups of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples.

The term “amount” or “level” of RNA expressed by a gene refers to the quantity of copies of an RNA transcript being assayed, including fragments of full-length transcripts that can be unambiguously identified as fragments of the transcript being assayed. Such quantity may be expressed as the total quantity of the RNA, in relative terms, e.g., compared to the level present in a control RNA sample, or as a concentration e.g., copy number per milliliter, of the RNA in the sample. The amount be of DNA molecules that are naturally fragmented (referred to as cell-free DNA) or that are fragmented by an artificial process (e.g., sonication or via an enzyme) that is applied to cellular DNA.

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A tumor-derived nucleic acid can refer to any nucleic acid released from a tumor cell, including pathogen nucleic acids from pathogens in a tumor cell. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed.

As used herein, the term “expression level” of a gene as described herein refers to the amount of an RNA transcript, e.g., an mRNA transcript, of the gene.

The terms “host gene expression” as used in this disclosure in the context of a gene expression panel, refers to the amount of RNA in a nucleic acid sample from a subject that is expressed by a gene originating from the host, i.e., the subject, as opposed to expression of a microbial, e.g., bacterial, viral, or fungal, gene.

Human genes are typically referred to herein using the official symbol and official nomenclature for the human gene as assigned by the HUGO Gene Nomenclature Committee, when HUGO nomenclature is available. In the present disclosure, an individual gene as designated herein may also have alternative designations, e.g., as indicated in the HGNC database. As used herein, the term “signature gene” refers to a gene whose expression is correlated with LRTI. A “gene panel” refers to a collection of such signature genes for which gene expression scores are generated and used to provide a risk/likelihood score for LRTI. Reference to the gene by name includes any human allelic variant or splice variant encoded by the gene.

The term “nucleic acid” or “polynucleotide” as used herein refers to a deoxyribonucleotide or ribonucleotide in either single- or double-stranded form. In the context of primers or probes, the term encompasses nucleic acids containing known analogues of natural nucleotides which have similar or improved binding properties, for the purposes desired, as the reference nucleic acid; and nucleic-acid-like structures with synthetic backbones.

The terms “identical” or percent “identity,” in the context of two or more nucleic acids or polypeptide sequences, refer to two or more sequences or subsequences that are the same (“identical”) or have a specified percentage of amino acid residues or nucleotides that are the same (i.e., at least about 70% identity, at least about 75% identity, at least 80% identity, at least about 90% identity, preferably at least about 91%, 92%, 93%, 94%, 95%, 96%, 97%, 98%, 99%, or higher identity over the entire sequence of a specified region, when compared and aligned for maximum correspondence over a comparison window or designated region. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). Algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (www.ncbi.nlm.nih.gov/).

The term “treatment,” “treat,” or “treating” typically refers to a clinical intervention, including multiple interventions over a period of time, to ameliorate at least one symptom of LRTI or otherwise slow progression. This includes alleviation of symptoms or diminishment of any direct or indirect pathological consequences of LRTI.

A “machine learning model” can refer to a software module configured to be run on one or more processors to provide a classification or numerical value of a property of one or more samples. An example type of model is supervised learning that can be used with embodiments of the present disclosure. Example supervised learning models may include different approaches and algorithms including analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm. The model may include linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

### DETAILED DESCRIPTION

Certain embodiments described herein includes analyzing nucleic acids of a biological sample of a subject to determine the likelihood of lower-respiratory tract infection (LRTI) in the subject. In particular, the analysis includes three methods for determining the likelihood of LRTI in the subject: (a) a host-based classifier; (b) a diversity model for analyzing nucleic acids corresponding to pathogens; and (c) an integrated meta-classifier that incorporates the host-based classifier and the diversity model.

In some embodiments, the host-based classifier includes predicting the likelihood of LRTI in the subject based on analyzing profiling RNA of host marker genes that are associated with LRTI. The RNA of the subject can be detected from each member of a gene panel, in which each gene member is identified as being differently expressed in subjects with LRTI compared to control subjects. For example, GNLY that encodes an anti-bacterial peptide in cytotoxic T Cells can be a member of the gene panel, since the expression of GNLY is higher in subjects with LRTI compared to control subjects. In another example, FABP4 that encodes a fatty acid-binding protein considered a marker of alveolar macrophages can be a member of the gene panel, since the expression of FABP4 is lower in subjects with LRTI compared to control subjects. In some instances, the gene panel includes at least two members selected from a group consisting of GNLY, PSMB8, FFAR3, SLC38A2, ISG15, IRF1, KIAA1841, AC090425.2, AKR1C3, CXCL5, SESN1, PCOLCE2, RBP4, TAP1, EPSTI1, and FABP4. In other instances, the gene panel includes at least 3, 4, 5, 6, 7, 8, 9, 10, 11, 12, 13, 14, 15, or all of the members of this group. The members of the gene panel can be selected by applying lasso logistic regression to training samples.

From the detected RNA, a quantity of differential gene expression for each member of the gene panel can be determined. The determined quantity of differential gene expression can be relative to the reference levels of RNA in control subjects. The respective quantities of differential gene expression can be used to determine a probability value, in which the probability value is indicative of whether the subject has an increased likelihood of LRTI, e.g., relative to subjects with the probability value being below the threshold value. The probability value can be generated by applying a machine-learning model (e.g., a random forest model) to the respective quantities of differential gene expression. Additionally or alternatively, the probability value can be a weighted sum of the respective quantities of differential gene expression. Once the probability value is determined, the subject can be determined as having an increased likelihood of LRTI based on the probability value exceeding a threshold value. If the probability exceeds the threshold value, the patient can be treated for LRTI or additional diagnostic tests can be performed.

The diversity model for detecting pathogens can be used to analyze nucleic acids pathogens in the biological sample to determine whether the subject has an increased likelihood of LRTI, e.g., relative to subjects having a different parameter value. The nucleic acids originating from a plurality of microbial species can be detected from the biological sample. These nucleic acids can be detected by obtaining sequence reads of the biological sample, aligning the sequence reads of the biological sample to one or more microbial reference genomes, and identifying aligned sequence reads as the nucleic acids that originate from the plurality of microbial species. For each microbial species of the plurality of microbial species, a nucleic-acid abundance level can be determined from the detected nucleic acids. For example, the nucleic-acid abundance level can include determining reads-per-million (RPM) values of nucleic acids that correspond to a respective microbial species. Then, a parameter can be determined based on the nucleic-acid abundance levels of the plurality of microbial species. In some instances, the parameter identifies an extent of microbial diversity in the biological sample. For example, the parameter can be a normalized diversity index (e.g., Shannon diversity index, Simpson diversity index). In another example, the parameter can be determined based on the nucleic acids corresponding to one or more microbial species that have nucleic-acid abundance levels that exceed a gap threshold. The parameter can then be compared to a threshold. If the parameter is below the threshold, it can be determined that the subject has an increased likelihood of LRTI.

In one aspect, the above techniques can be combined into an integrated meta-classifier to determine whether the subject has an increased likelihood of LRTI, e.g., relative to other subjects having different values, such as a probability value and a parameter value. In particular, the host-based classifier can be applied to the RNA of the subject to generate a probability value of whether the subject has an increased risk of having LRTI, in which the RNA corresponds to members of the gene panel identified as being differently expressed in subjects with LRTI compared to control subjects (e.g., GNLY). The diversity model can be applied to nucleic acids (e.g., DNA, RNA) originating from microbial species to generate a parameter indicative of whether the subject has an increased risk of having LRTI. In addition to the two values, another parameter can be generated based on abundance level of nucleic acids that originate from a plurality of virus species. Based on the three values, a final output can be determined that determines whether the subject has an increased likelihood of having LRTI. In some instances, a logistical regression model is applied to the three values to generate the final output. The combination of host gene expressions and microbial profiling enables accurate LRTI diagnosis and pathogen identification in critically-ill subjects.

### I. Overview

LRTI involves a dynamic relationship between pathogen, lung microbiome and host response that is generally not captured by existing clinical diagnostic tests. In particular, incidental carriage of pathogens in the respiratory tract is common in pediatric patients. However, detection of a pathogen using mNGS alone was often insufficient for accurate LRTI diagnosis in pediatric cohorts. For example, among pediatric patients that was predicted as having no evidence of LRTI using mNGS, 40% of the patients still had potentially pathogenic microbes. Such finding was notably different from adults, for whom prior metagenomic studies have demonstrated much lower rates of incidental pathogen carriage. Thus, additional techniques (e.g., profiling the host response) were needed for accurate pediatric LRTI diagnosis.

To address this deficiency, metagenomic analysis of lower respiratory samples can be used to detect LRTI and identify features that contribute to accurate LRTI diagnosis. The present techniques can be used for particular vulnerable pediatric population, a demographic facing a high burden of LRTI.

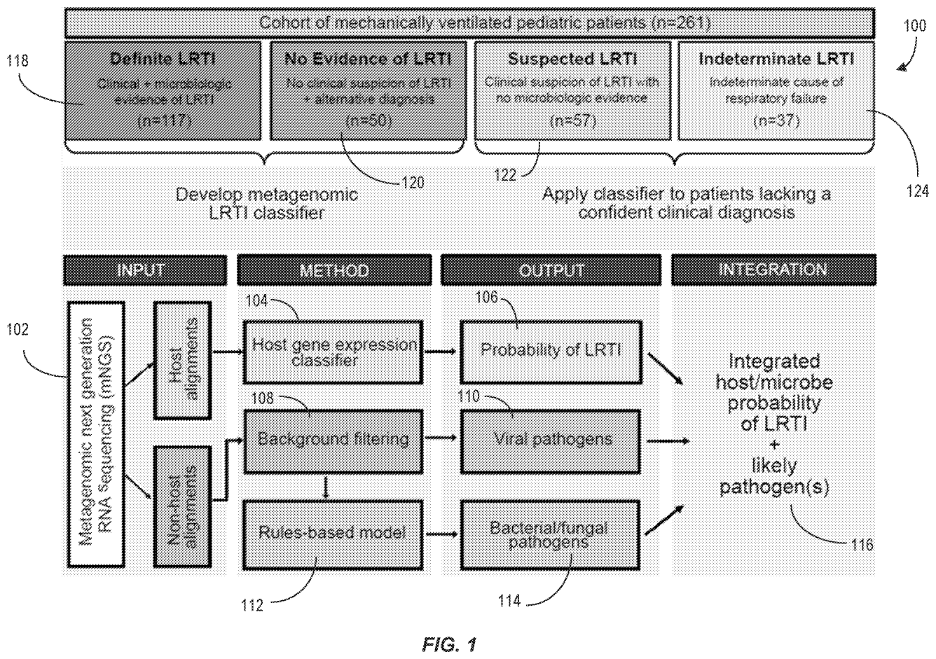

FIG. 1 shows a schematic diagram that illustrates a process 100 for determining a likelihood of LRTIs in subjects, according to some embodiments. A biological sample (e.g., lower respiratory fluid sample) including a mixture of nucleic acids from the subject and microbes can be obtained. The mixture of nucleic acids can include RNA, DNA, or both. In some instances, the mixture of nucleic acid molecules of the biological sample is sequenced (e.g., using NGS) to generate a plurality of sequence reads (block 102). A first set of nucleic acid molecules that originate a human subject (e.g., sequence reads that align to the human reference genome) can be used by a host gene-expression classifier (block 104), while a second set of nucleic acid molecules that do not originate from the human subject (e.g., sequence reads that do not align to the human reference genome) can be used for microbial analysis.

A host classifier can determine, based on gene expression of the first set of nucleic acids for genes of a particular gene panel, a probability value that indicates the subject has an increased likelihood of LRTI (block 106). The members of the gene panel can be selected from activation markers of T cells, alveolar macrophages and the interferon response, which successfully captures cases of viral infection, bacterial infection or co-infection. Additionally or alternatively, the host classifier can predict whether there is an increased likelihood of the subject having an LRTI, based on host gene expression levels corresponding to six genes that exhibit the most discriminating power. The host classifier performed accurately, with a median AUC of 0.967 by cross-validation. The accurate performance of the host classifier thus suggests that host gene expression alone could be effective and can be incorporated into a clinical PCR assay as a standalone rapid diagnostic. The host classifier can be trained based on training data obtained from a cohort that includes two groups: (i) a first group of patients diagnosed with LRTI (“Definite”); and (ii) a second group of patients having no evidence of LRTI (“No Evidence”).

For the second set of nucleic acids that do not originate from the human subject, a set of microbial analyses can be performed. First, a background filtering can be performed to identify nucleic acids that originate from a plurality of virus species (block 108). The identified nucleic acids can be used to determine an abundance level of the nucleic acids that originate from the plurality of virus species. In some instances, the filtered nucleic acids are aligned to one or more virus genomes to determine types of virus species with which the nucleic acids are associated (block 110).

The remaining nucleic acid molecules can then be used for determining whether bacterial or fungal pathogens contribute to the occurrence the LRTI in the human subject. The diversity model for detecting pathogens can be used to analyze nucleic acids pathogens in the biological sample to determine whether the subject has an increased likelihood of LRTI (block 112). For example, for each microbial species of a plurality of microbial species, a nucleic-acid abundance level can be determined from the detected nucleic acids (block 114). The nucleic-acid abundance level can include determining reads-per-million (RPM) values of nucleic acids that correspond to a respective microbial species. In some instances, a parameter is determined based on the nucleic-acid abundance levels of the plurality of microbial species, in which the parameter identifies an extent of microbial diversity in the biological sample. The parameter can be determined based on the nucleic acids corresponding to one or more microbial species that have nucleic-acid abundance levels that exceed a gap threshold. The parameter can then be compared to a threshold. If the parameter is below the threshold, it can be determined that the subject has an increased likelihood of LRTI.

To further enhance the performance and to detect incidental carriage of pathogens by pediatric subjects, the results generated by the host classifier (e.g., probability value) can be integrated with the microbial features such as abundance levels of respiratory viruses in the biological sample and a relative dominance of bacteria/fungi deemed potential pathogens (block 116). The three features can be processed using a logistic regression model to predict whether the subject has an increased likelihood of LRTI. The integrated host/microbe classifier achieved a median AUC of 0.986 by cross-validation. The incorporation of microbial features thus significantly increased the confidence of LRTI classification. Based on a comparison with the results generated by the host classifier, relatively few patients switched from their predicted diagnosis. However, the output probabilities generated by the integrated classifier were more definitive compared to the outputs generated by the host-only classifier, thereby providing more confidence of the diagnosis. It is likely that the integrated classification approach will prove even more valuable in settings where the host gene expression may not perform as well on its own (e.g., immune-compromised patients), and will generalize better to future cohorts. Moreover, the integrated classifier can provide clinicians with a unified framework both for LRTI diagnosis and pathogen identification.

In contrast to host gene expression, associating microbial features with LRTI diagnosis can be challenging given the sparse presence of individual respiratory pathogens across patients in the cohort, especially in the groups that were initially classified as not having an LRTI. In some instances, larger datasets are generated to implement machine learning approaches to capture the null distribution of incidentally carried pathogens in the lower respiratory tract and identify outlier cases that signal LRTI. Even when using larger datasets, designating a specific microbe as a ‘true’ causal pathogen for training purposes would be non-trivial, especially for subjects having co-infection. To address the above challenges, a different technique can be used, in which features relating to a collapse of lung microbiome diversity can be used as an established feature of detecting a likelihood of LRTI in subjects. The collapse of lung microbiome can be indicated by detecting a presence of a dominant pathogen in the biological sample.

An advantage of incorporating the present techniques is the capacity to provide a microbiologic diagnosis when traditional clinical testing returns negative results, as in an estimated 30-60% of suspected community- or hospital-acquired pneumonia cases. The integrated classifier was able to confirm LRTI in 65% of children in the cohort having a suspected infection but initially diagnosed in negative during clinical testing. The integrated classifier was able to confirm LRTI in 32% of patients with respiratory failure but having an indeterminate etiology.

In some instances, the integrated classifier also provided a microbiologic diagnosis in all but one of the above patients, highlighting the potential to inform a treatment that can be effectively used for the patients (e.g., pathogen-targeted treatment, empirical treatment). Acute respiratory illnesses can be a leading contributor to inappropriate antimicrobial use, a practice driven by challenges distinguishing LRTI from non-infectious causes of respiratory failure. Reflecting this is the observation that 90% of children in the cohort received empiric antimicrobials by the time of sample collection, including 84% in the No Evidence group. To minimize the occurrence of the inappropriate antimicrobial use, the integrated classifier can provide an opportunity for an improved determination of whether antimicrobial treatments can be used, particularly in clinically in subjects with uncertain diagnoses. In particular, the integrated classifier can determine a probability of LRTI, to inform a clinician as to whether the antimicrobial treatments should be applied. Additionally or alternatively, the integrated classifier can also be used to predict the pathogen species (e.g., RSV) causing the LRTI, at which a particular type of antimicrobial or antiviral treatment (e.g., RSV monoclonal antibody) can be selected based on the predicted pathogen species. The integrated classifier can also be tuned to achieve >98% sensitivity for LRTI detection, highlighting its potential use as a rule-out test to help exclude the need for antimicrobials. In addition to the integrated classifier, a host classifier alone (without microbial features) can be used specifically for detecting bacterial infection, which could also inform the need for antibiotics usage.

The present techniques for diagnosing LRTI can be used at different time points of microbial infection, including the time of intubation for critically-ill children with acute respiratory failure. The present techniques can be used to diagnose LRTI without a need for bacterial culture test. Additionally or alternatively, the present techniques can be used as a complement to traditional culture and PCR-based microbiologic testing. Accordingly, combining host gene expression and unbiased microbial profiling from lower airway mNGS enables accurate LRTI diagnosis and pathogen identification in critically-ill children.

### II. Determining Classification of LRTI Based on Differential Gene Expression

To obtain the biological samples, child patients with acute respiratory failure and requiring mechanical ventilation were enrolled. Tracheal aspirate (TA) samples of the eligible subjects were collected within 24 hours of intubation and underwent metagenomic analysis of RNA to assay host gene expression and detect respiratory microbiota. As a result, high-quality host gene expression and microbial data was obtained for the subjects eligible for the study.

Adjudication of LRTI status was carried out according to a final clinical diagnoses assigned by treating physicians and standard-of-care microbiologic diagnostics performed at each study site, consisting of nasopharyngeal (NP) swab viral PCR and TA culture.

Referring back to FIG. 1, the subjects were classified into different groups for further analysis. For example, a first group of subjects 118 (“Definite LRTI”) received an LRTI diagnosis and positive microbiologic findings were identified. A second group of subjects 120 (“No Evidence of LRTI”) were identified to a clear non-infectious cause of acute respiratory failure and no clinical or microbiologic suspicion of LRTI. A third group of subjects 122 (“Suspected LRTI”) received an LRTI diagnosis, but the microbiologic tests returned negative results. A fourth group of subjects 124 (“Indeterminate LRTI”) were considered as being uncertain as to whether LRTI was a contributing factor to respiratory failure due to conflicting clinical and microbiologic findings.

For each group of subjects, gene expression for each of a plurality of genes can be measured. Then, a set of genes that exhibit a statistically significant difference of expression levels between LRTI and non-LRTI subjects can then be selected. For example, genes such as GNLY, PSMB8, FFAR3, SLC38A2, ISG15, IRF1, KIAA1841, AC090425.2, AKR1C3, CXCL5, SESN1, PCOLCE2, RBP4, TAP1, EPSTI1, and FABP4 show a statistically significant difference of expression levels between LRTI and non-LRTI subjects. For example, FABP4 is a fatty acid binding protein that, based on the DE analysis, appears to be a strong biomarker of noninfectious pulmonary inflammation which is significantly expressed in subjects who have severe respiratory illnesses but do not have LRTIs. The differentially expressed genes can also correspond to genes that are known to be associated with inflammatory signaling in the context of infection (e.g., alpha and data signaling pathways).

The set of genes (or a subset thereof) can then be used as a gene panel for determining a likelihood of LRTI in a given subject. For example, RNA obtained from the subject's biological sample can be analyzed to determine a quantity of differential gene expression for each member of the gene panel compared to reference levels of RNA in control subjects. Based on the respective quantities of different gene expression, a probability value indicative of a likelihood of LRTI can be determined for the subject.

### A. Cohort Selection

A prospective cohort of mechanically ventilated children admitted to eight Pediatric Intensive Care units in the National Institute of Child Health and Human Development's Collaborative Pediatric Critical Care Research Network (CPCCRN) from February 2015 to December 2017 was selected. The study was approved by the single Collaborative Pediatric Critical Care Research IRB at the University of Utah (protocol #00088656). Informed consent was obtained from parents or other legal guardians, which included permission for collected specimens and data to be used in future studies.

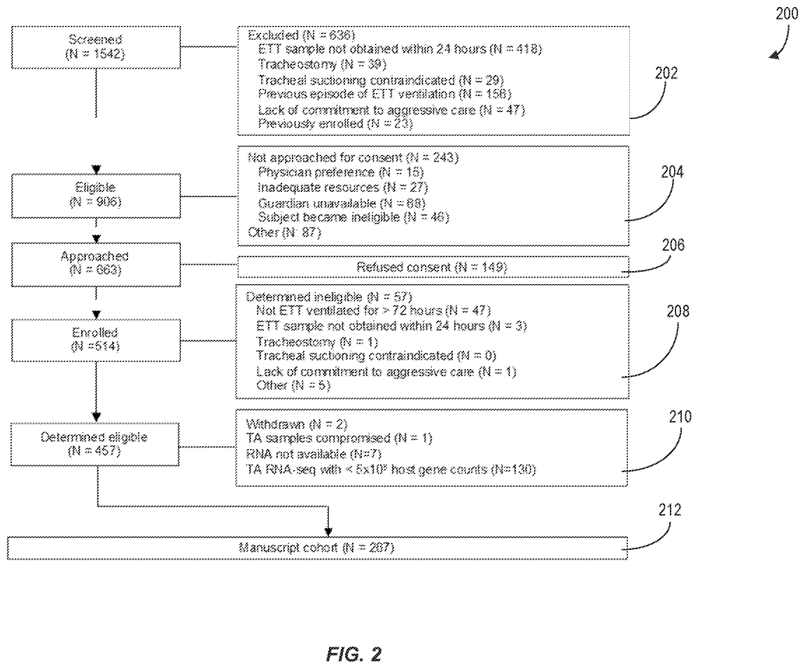

FIG. 2 shows a schematic diagram that illustrates a screening process 200 for selecting a cohort for determining a likelihood of LRTIs in subjects, according to some embodiments. Initially, a total number of 1542 subjects were initially selected for the screening process (block 202), in which the subjects included children aged 31 days to 18 years who were expected to require mechanical ventilation (MV) via endotracheal tube (ETT) for at least 72 hours.

From the total number of subjects, 906 eligible subjects were selected (block 204). For example, some subjects were excluded for this study, due to physician decision, unavailability of a guardian, etc. In particular, exclusion criteria included inability to obtain a TA sample from the subject within 24 hours of intubation; presence of a tracheostomy tube or plans to place one; any condition in which deep tracheal suctioning was contraindicated; previous episode of MV during the hospitalization; family/team lack of commitment to aggressive intensive care as indicated by ‘do not resuscitate’ orders and/or other limitation of care; or previous enrollment into this study. Some patients were ultimately excluded from the present analysis based on sequencing metrics.

From the group of eligible subjects, 663 subjects consented to the screening process (block 206). Parents or other legal guardians of eligible patients were approached for consent by study-trained staff as soon as possible after intubation. Waiver of consent was granted for TA samples to be obtained from standard-of-care suctioning of the ETT until the parents or guardians could be approached for informed consent.

The consented subjects were enrolled (block 208) and screened (block 210) to determine a cohort of 267 subjects (block 212). The subjects in the cohort received standard-of-care clinical respiratory microbiologic diagnostics, as ordered by treating clinicians at each study site. These diagnostics included nasopharyngeal (NP) swab respiratory viral testing by multiplex PCR and/or tracheal aspirate (TA) bacterial and fungal semi-quantitative cultures. Clinical diagnostic tests on samples obtained within 48 hours of intubation were included in the analyses. Microbes reported by the clinical laboratory as representing laboratory, skin or environmental contaminants, or reported as mixed upper respiratory flora, were excluded.

Clinical data were then collected from the cohort and recorded in a web-based research database maintained by the CPCCRN data coordinating center at the University of Utah.

### B. Adjudication of LRTI

Adjudication of LRTI statuses were determined for each subject of the cohort. The adjudication of LRTI was based on the final diagnoses reported by treating clinicians at each study site, who typically reviewed chest x-ray findings as part of this process. The final diagnoses also included any standard-of-care clinical respiratory microbiologic diagnostics performed during the admission.

The subjects of the cohort were ultimately assigned into one of four groups by study team physicians who were blinded to the mNGS results: (i) Definite, in which clinicians established an LRTI diagnosis and the patient had positive microbiologic findings; (ii) Suspected, in which clinicians established an LRTI diagnosis but the microbiologic testing performed returned negative; (iii) Indeterminate, in which it remained uncertain whether LRTI was a contributing factor to respiratory failure due to conflicting clinical and microbiologic findings; and (iv) No Evidence, in which clinicians identified a clear non-infectious cause of acute respiratory failure and no clinical or microbiologic suspicion of LRTI arose. Some of the No Evidence subjects did not have comprehensive clinical microbiologic tests performed, due to the absence of clinical suspicion.

Table 1 provides the following demographics and clinical characteristics of the cohort determining based on the process 200 of FIG. 2. The determination of LRTI or No-LRTI for the subjects was based on clinical data collected during or after their hospitalization.

TABLE 1
Demographics and clinical characteristics.

| | Definite LRTI | | No-LRTI | | | Suspected LRTI | | Indeterminate | |
|---|---|---|---|---|---|---|---|---|---|
| | (n = 117) | | (n = 50) | | p value* | (n = 57) | | LRTI (n = 43) | |
| Female, n (%) | 45 | (38.5%) | 25 | (50.0%) | 0.18 | 26 | (45.6%) | 20 | (46.5%) |
| Age, median [IQR] | 0.5 | [0.2, 1.8] | 6.5 | [1.5, 12.9] | <0.001 | 1.7 | [0.5, 6.0] | 1.45106092 | |
| | | | | | | | | [symbol] | |
| Race, n (%) | | | | | | | | | |
| White | 69 | (59.0%) | 30 | (60.0%) | 0.99 | 33 | (57.9%) | [symbol] | |
| Black/African American | 26 | (22.2%) | 7 | (14.0%) | 0.29 | 11 | (19.3%) | 10 | (23.3%) |
| Asian | 5 | (4.3%) | 6 | (12.0%) | 0.088 | 2 | (3.5%) | 2 | (4.7%) |
| American Indian or Alaskan Native | 1 | (0.9%) | 1 | (2.0%) | 0.99 | 1 | (1.8%) | 0 | (0.0%) |
| Native Hawaiian/Other Pacific | 1 | (0.9%) | 0 | (0.0%) | 0.99 | 0 | (0.0%) | 1 | (2.3%) |
| Islander | | | | | | | | | |
| More than one race | 3 | (2.6%) | 1 | (2.0%) | 0.99 | 1 | (1.8%) | 2 | (4.7%) |
| Unknown | 12 | (10.3%) | 5 | (10.0%) | 0.99 | 9 | (15.8%) | 6 | (14.0%) |
| Hispanic or Latino, n (%) | 17 | (14.5%) | 6 | (12.0%) | 0.81 | 14 | (24.6%) | 11 | (25.6%) |
| Comorbidities (CCC)†, n (%) | 38 | (32.5%) | 26 | (52.0%) | 0.024 | 34 | (59.7%) | 18 | (41.9%) |
| Immunosuppressed, n (%) | 3 | (2.6%) | 7 | (14.0%) | 0.0085 | 5 | (8.8%) | 6 | (14.0%) |
| Admission category, n (%) | | | | | | | | | |
| Medical | 117 | (100.0%) | 28 | (56.0%) | p < 0.001 | 57 | (100.0%) | 35 | (81.4%) |
| Surgical | 0 | (0.0%) | 15 | (30.0%) | p < 0.001 | 0 | (0.0%) | 3 | (7.0%) |
| Trauma | 0 | (0.0%) | 7 | (14.0%) | p < 0.001 | 0 | (0.0%) | 4 | (9.3%) |
| Time from hospital admission to | 4.8 | [0.0, 23.6] | 3.5 | [0.0, 20.9] | 0.60 | 2.6 | [0.0, 15.9] | 1.7 | [0.0, 47.4] |
| intubation (hours), median [IQR] | | | | | | | | | |
| PRISM III#, median [IQR] | 3.0 | [0.0, 6.0] | 8.0 | [3.3, 11.8] | <0.001 | 6.0 | [2.0, 11.0] | 8.0 | [3.0, 13.0] |
| Antibiotics before sample§, n(%) | 112 | (95.7%) | 42 | (84.0%) | 0.022 | 51 | (89.5%) | 30 | (69.8%) |

In Table 1, “*” indicated that the p value was determined based on a comparison between the Definite LRTI and No-LRTI cases, in which Wilcoxon rank sum test was used for all continuous variables and Fisher’s exact test was used for all categorical variables. In addition, “†” indicated complex chronic conditions, “‡” indicated Pediatric Risk of Mortality Score, and “§” indicated that Antibiotic treatment was started on or before the day of sample collection. [symbol]
indicates data missing or illegible when filed

In Table 1, “*” indicated that the p value was determined based on a comparison between the Definite LRTI and No-LRTI cases, in which Wilcoxon rank sum test was used for all continuous variables and Fisher's exact test was used for all categorical variables. In addition, “†” indicated complex chronic conditions, “‡” indicated Pediatric Risk of Mortality Score, and “†” indicated that Antibiotic treatment was started on or before the day of sample collection.

As shown in Table 1, the subjects in the Definite group were 39% female with a median age of 0.5 years (IQR 0.2-1.8), and the patients in the No Evidence group were 50% female with a median age of 6.5 years (IQR 1.5-12.9). The difference in the age distribution of the two groups (p<0.001, Wilcoxon rank-sum test) reflected recognized epidemiological distinctions in the conditions typically leading to respiratory failure in very young versus older children. Diagnoses in the No Evidence group included trauma, neurological conditions, cardiovascular disease, airway abnormalities, ingestion of drugs/toxins, and sepsis that was clearly unconnected to LRTI. It is noted from the clinical data that most subjects received antibiotic treatment by the time of tracheal-aspirate sample collection in both the Definite (96%) and No Evidence (84%) groups, regardless of their respective diagnoses.

In addition to the Definite and No Evidence groups, two other categories were identified (e.g., suspected LRTI, indeterminate LRTI). The subjects in the two other categories can include those who were suspected of having LRTI but had a negative culture test result.

Further, within the Definite group, clinical microbiologic testing identified viral infection alone in 46% of patients, bacterial infection alone in 14% of patients, and viral/bacterial co-infection in 40% of patients. The most common pathogens were respiratory syncytial virus (RSV) and Haemophilus influenzae, which frequently were found together in the same biological sample.

C. Sample Collection, Processing, and mNGS

After the subjects were divided into four LRTI groups, sequence data were generated for each subject of the cohort. The sequence data were then used to determine a correlation between DNA and/or RNA of the subject and a likelihood of the LRTI.

TA sample for each subject was collected within 24 hours of intubation. The TA sample was mixed 1:1 with DNA/RNA Shield (Zymo) and frozen at −80° C. RNA was extracted from 300 μl of the TA sample using bead-based lysis and the Allprep DNA/RNA kit (Qiagen), which included a DNase treatment step. RNA was reverse transcribed to generate cDNA, and sequencing library preparation was performed using the NEBNext Ultra II Library Prep Kit. RNA-Seq libraries underwent 150 nucleotide paired-end sequencing on an Illumina Novaseq 6000 instrument.

### D. Host Gene Expression Analysis

Following de-multiplexing, sequencing reads were pseudo-aligned with kallisto (v. 0.46.1; including bias correction) to an index consisting of all transcripts associated with human protein coding and long non-coding RNA genes (ENSEMBL v.99). TA samples with less than 500,000 estimated counts associated with transcripts of protein-coding genes were excluded. Gene-level counts were generated from the transcript-level abundance estimates using the R package tximport, with the scaledTPM method.

Genes were retained for differential expression (DE) analysis if they had at least 10 counts in at least 20% of the TA samples included in the analysis. DE analyses were performed with the R package limma, using quantile normalization and the voom method. Gene set enrichment analyses (GSEA) were performed using the fgseaMultilevel function in the R package fgsea on REACTOME pathways with a minimum size of 10 genes and a maximum size of 1,500 genes. All genes from the respective DE analysis were included as input, pre-ranked by the DE test statistic. The gene sets shown in the figures were manually selected to reduce redundancy and highlight diverse biological functions from among those with a Benjamini-Hochberg adjusted p-value<0.05.

Based on results of the DE analysis, host gene expression between the Definite and No Evidence groups were first compared to determine whether it could distinguish patients based on LRTI status.

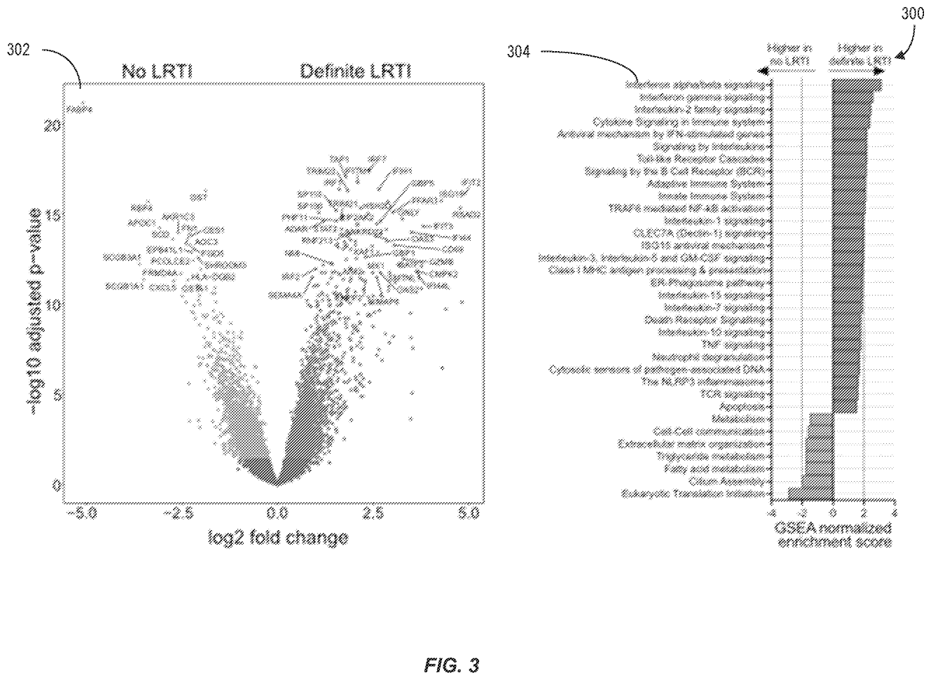

FIG. 3 shows an example set of graphs 300 illustrating a correlation of gene expression and classification of LRTI status, according to some embodiments. In FIG. 3, a volcano plot 302 identifies genes differentially expressed (DE) between Definite and No Evidence subjects. The volcano plot 302 further includes colors for genes that reached statistical significance (adjusted p-value<0.05). As shown in the volcano plot 302, 4,718 differentially expressed genes were identified at a Benjamini-Hochberg adjusted p-value<0.05. The p-value was relative to the subjects with non-infectious respiratory failure (i.e., No Evidence).

With respect to the GSEA analysis, FIG. 3 shows a set of normalized enrichment scores 304 of selected REACTOME pathways that reached statistical significance (adjusted p-value<0.05) in the GSEA using DE genes between Definite and No Evidence groups. The GSEA analysis was performed to identify biological relevance of differentially expressed genes in subjects. The GSEA analysis can be performed by mapping the list of differentially expressed genes (e.g., genes selected based on log 2 fold change difference in expression between LRTI and No Evidence groups) to other genes known to be associated with certain biological signaling pathways.

For example, there can be approximately 200 genes known to be associated with interferon alpha and beta signaling pathways. If the list of differentially expressed genes includes 180 genes out of those 200 known genes, then a high enrichment score corresponding to the interferon signaling pathway can be identified. Thus, the enrichment score corresponds to an algorithmic score of all the genes related to that a corresponding signaling pathway. And the enrichment score can be compared with other enrichment scores of other signaling pathways to determine which biological signaling pathways are affected in subjects with LRTI.

As shown in the normalized enrichment scores 304, the GSEA identified elevated expression of pathways involved in the immune response to infection in the Definite group. Further, pathways related to the interferon response, a hallmark of anti-viral innate immunity, were most strongly upregulated, consistent with the high prevalence of viral infections in the Definite group. Additional immune pathways upregulated in this group included toll-like receptor signaling, cytokine signaling, inflammasome activation, neutrophil degranulation, antigen processing, and B cell and T cell receptor signaling. Conversely, the normalized enrichment scores 304 also show pathways with reduced expression in the Definite group, in which the pathways included translation, cilium assembly and lipid metabolism.

Based on the volcano plot 302 and the normalized enrichment scores 304, a set of genes that exhibit a statistically significant difference of expression levels between LRTI and non-LRTI subjects can then be selected. For example, genes such as GNLY, PSMB8, FFAR3, SLC38A2, ISG15, IRF1, KIAA1841, AC090425.2, AKR1C3, CXCL5, SESN1, PCOLCE2, RBP4, TAP1, EPSTI1, and FABP4 show a statistically significant difference of expression levels between LRTI and non-LRTI subjects. For example, FABP4 is a fatty acid binding protein that, based on the DE analysis, appears to be a strong biomarker of noninfectious pulmonary inflammation which is significantly expressed in subjects who have severe respiratory illnesses but do not have LRTIs. The differentially expressed genes can also correspond to genes that are known to be associated with inflammatory signaling in the context of infection (e.g., alpha and data signaling pathways). In some instances, a smaller subset of genes is selected, which includes GNLY, PSMB8, FFAR3, SLC38A2, ISG15, IFR1, RBP4, and FABP4.

The identification of differentially expressed genes can then be used to classify whether a given subject has LRTI. For example, elevated expression levels of GNLY, PSMB8, FFAR3, SLC38A2, ISG15, IFR1, RBP4, and FABP4 of the subject can be predictive of a likelihood that the subject has LRTI.

### E. Method

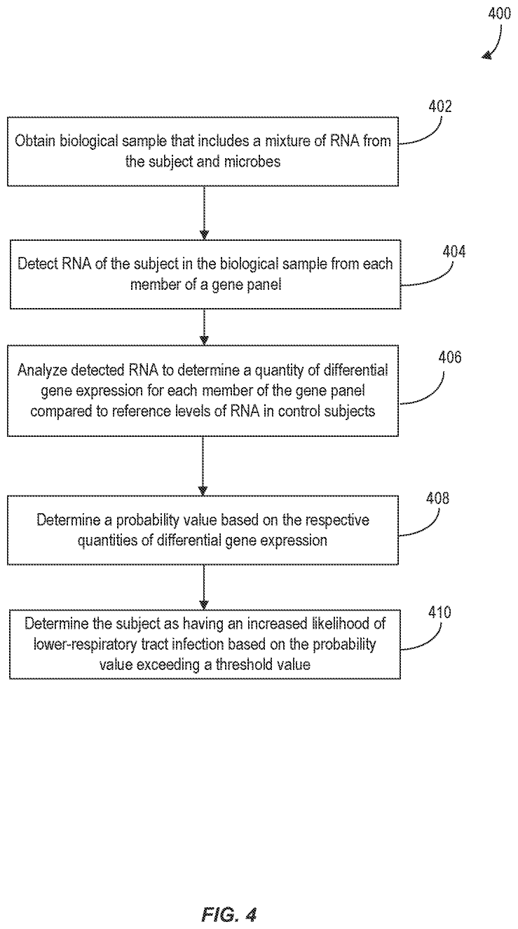

FIG. 4 is a flowchart for a method 400 for determining a likelihood of LRTI in a subject based on gene expression levels, according to some embodiments. At least a portion of the method may be performed by a computer system.

At block 402, a biological sample of a subject is obtained. The biological sample can include a mixture of RNA from the subject and microbes. Exemplary biological samples are described herein and include those obtained, for example, by a nasal swab, nasopharyngeal swab, nasopharyngeal wash or aspirate, mid-turbinate nasal swab, oropharyngeal swab, buccal swab, a broncho-alveolar lavage, or an endotracheal aspirate. In some embodiments, the biological sample is serum, plasma, blood, or solid tissue. In some embodiments, a sample may be processed to provide or purify RNA of a particular nucleic acid molecule or fragment thereof.

At block 404, RNA of the subject in the biological sample from each member of a gene panel is detected. Gene expression levels may be determined using any suitable method. For example, RNA may be sequenced using sequencing methods such as next-generation sequencing, high-throughput sequencing, massively parallel sequencing, sequencing-by-synthesis, paired-end sequencing, single-molecule sequencing, nanopore sequencing, pyrosequencing, semiconductor sequencing, sequencing-by-ligation, sequencing-by-hybridization, RNA-Seq, Digital Gene Expression, Single Molecule Sequencing by Synthesis (SMSS), Clonal Single Molecule Array (Solexa), shotgun sequencing, Maxim-Gilbert sequencing, primer walking, and Sanger sequencing. Sequencing methods may comprise targeted sequencing, whole-genome sequencing (WGS), lowpass sequencing, bisulfite sequencing, whole-genome bisulfite sequencing (WGBS), or a combination thereof. Sequencing methods may include preparation of suitable libraries. Sequencing methods may include amplification of nucleic acids (e.g., by targeted or universal amplification, such as PCR). Gene expression may also be assessed by PCR, Loop-Mediated Isothermal Amplification (LAMP), Transcription-Mediated Amplification (TMA), Isothermal Amplification or other nucleic acid amplification assay.

In some instances, the gene panel includes at least two members from a group of genes identified in Table 3 (discussed below). For example, the gene panel can include at least two members (or more) selected from a group of genes consisting of GNLY, PSMB8, FFAR3, SLC38A2, ISG15, IRF1, KIAA1841, AC090425.2, AKR1C3, CXCL5, SESN1, PCOLCE2, RBP4, TAP1, EPSTI1, and FABP4. The gene panel may thus comprise one or more genes set forth in the tables (e.g., Tables 1-3) and any additional genes identified as being correlated with LRTI risk. In other instances, the gene panel includes at least 3, 4, 5, 6, 7, 8, 9, 10, 11, 12, 13, 14, or 15 genes of the group identified in Tables 1-3. Differential gene expression of at least one gene of the above panel relative to reference levels can be indicative of a likelihood of LRTIs in subjects.

As other examples, the gene panel can comprise at least two members selected from the group consisting of GNLY, PSMB8, FFAR3, SLC38A2, ISG15, IFR1, RBP4, and FABP4. As another example, the gene panel comprises at least two members selected from the group consisting of TAP1, FABP4, RBP4, EPSTI1, and FFAR3. As another example, the gene panel comprises at least two members selected from the group consisting of TAP1, FABP4, and RBP4

At block 406, the detected RNA is analyzed to determine a quantity of differential gene expression for each member of the gene panel compared to reference levels of RNA in control subjects. The quantity of the differential gene expression can include quantity and/or the frequency of RNA present in the biological sample as compared to reference levels in control subjects. In effect, each member of the gene panel (e.g., GNLY) can be associated with a corresponding quantity of gene expression, thereby obtaining respective quantities of differential gene expression. In some instances, the quantity of differential expression for each member is determined using a difference or ratio between a measured expression level and a reference level. The process for measuring gene expression levels is further described in Section VI of the present disclosure.

In some instances, control subjects correspond to subjects having a clear non-infectious cause of acute respiratory failure and no clinical or microbiologic suspicion of LRTI. For example, a control subject can include a subject having infection at another location that was causing acute respiratory failure. A control population can include at least 10 subjects, 20 subjects, 30 subjects, 40 subjects, 50 subjects, or more than 50 subjects (e.g., 100 subjects). In some embodiments, a control population comprises 500 or more subjects.

At block 408, a probability value based on the respective quantities of differential gene expression is determined. The probability value can correspond to a predicted likelihood of the subject of having an LRTI. A relationship between the respective quantities and the likelihood (probability) of having LRTI can be determined, e.g., using a proportion of samples having LRTI that have a given quantity of differential expression.

The probability value can be determined based on a total quantity of differential expression, including a weighted sum or average of the individual quantities of differential expression. The weights can be based on the importance (discriminating power) of each marker in discriminating LRTI from non-LRTI. Then, the proportion of the subjects (i.e., training/reference subjects) that have LRTI at a given value (or within a certain range around the given value) for the total quantity can be used as the probability value. Accordingly, clusters of reference subjects, for which a classification of LRT1 has been confirmed at time of measurement or at a later time using a more time consuming or costly procedure, can be determined, with each cluster corresponding to a particular probability value, e.g., determined as a proportion of the reference subjects in the cluster that were classified as having the infection. Such a technique can be used with any of the methods described herein. As another example, a machine learning model can provide the probability, e.g., a random forest classifier can provide the probability value. The use of the machine-learning models to determine LRTI is further described in Section III of the present disclosure.

At block 410, the subject is determined as having an increased likelihood of lower-respiratory tract infection based on the probability value exceeding a threshold value. The threshold value can be selected based on a desired accuracy, e.g., a trade off of sensitivity and specificity. In some embodiments, likelihood of LRTI is assigned based on a cutoff value (also referred to as a threshold value) using a reference scale, e.g., from 0 to 1.0. In some embodiments, a cutoff value of 0.5 or greater may be employed to define likelihood of LRTI. In some embodiments, LRTI likelihood may be further stratified, for example, likelihood of LRTI may be categorized as “high,” “intermediate,” or “low”, e.g., based on the highest tertile, intermediate tertile and bottom tertile.

### III. Machine-Learning Techniques to Determine a Likelihood of LRT1 in Subjects

The analysis of host gene expression data described in Section II of the present disclosure can additionally include applying a machine learning model to distinguish between positive and negative LRTI samples based on the expression level of certain genes. The machine-learning model can be trained using a training set where the gene expression levels (acting as input features to the model) and known diagnosis (labels) that would distinguish between positive and negative LRTI samples (or between LRTI and other diseases). In the process of learning, the model identifies gene markers that are predictive for the disease state. In particular, a minimum gene set that was highly predictive at classifying the subjects as having or not having LRTIs can be identified. For example, a 14-gene signature can be identified by determining regression coefficients of each selected gene and the out-of-fold probability of LRTI assigned to each sample.

The host classifier can be used as part of a diagnostic test for determining whether the subject has LRTI. Based on the initial determination, additional metagenomic tests can be performed to identify pathogens causing the LRTIs for selecting an appropriate treatment for the subject (e.g., antibiotic treatments, antiviral treatments).

### A. Training Data

The training dataset included subjects from a cohort screened and selected using the processes described in FIGS. 1 and 2 of the present disclosure. For example, a cohort of pediatric patients can be selected and divided into the following subject groups: (i) Definite; (ii) Suspected; (iii) Indeterminate; and (iv) No Evidence. For patients of each group, AT samples can be collected and sequenced to generate RNA sequence reads. The RNA sequence reads can be aligned to all transcripts associated with human protein coding and long non-coding RNA genes, thereby identifying a set of genes associated with the biological samples.

From the set of genes, genes having at least 10 counts in at least 20% of the Definite (n=117) and No Evidence (n=50) subjects were selected and used as input for training the host classifier. The total amount of genes amounted to 13,323. A variance-stabilizing transformation was applied to the gene counts, as implemented in the R package DESeq2.

In some embodiments, different subsets of genes are selected to form a subset of training samples. This training subset can then be used to train (optimize) a model, whose accuracy can be measured, e.g., using the AUC of an ROC curve. Then, another subset of genes can be selected, with a further training process providing another model whose accuracy can also be measured. The accuracy can be measured using the training set or a validation set, which can include samples with known labels that were excluded from the training set. This process of generating models for different subsets of genes, along with the accuracy of each model, can continue, possibly for all possible subsets of genes for which expression levels have been measured. The subsets can be constrained to a specified number of host genes (e.g., 1 or 2).

### B. Model Configuration

The machine-learning model for the host classifier can be selected for determining a likelihood of a subject having an LRTI. In some instances, different machine-learning models are used, each one directed to a different type of classification. For example, a model can determine whether a subject having determine the likelihood of the subject having an LRTI. A further model can determine whether the subject has an increased mortality risk or not. A further model can classify a predicted response of a subject to a particular type of treatment. In some embodiments, supervised machine learning (e.g. decision trees, nearest neighbor, support vector machines, and neural networks) and/or unsupervised machine learning (e.g., clustering, principal component analysis, etc.) is used for the host classifier.

For example, a random forest classifier can be selected as the model for the host classifier for determining a likelihood of LRTI in subjects. The random forest classifier can be implemented using the R package random forest. For the model configuration, 10,000 trees were used and all parameters were left at their defaults.

Further examples of machine-learning algorithms include quadratic discriminate analysis, support vector machines, including without limitation support vector classification-based regression processes, stochastic gradient descent algorithms, nearest neighbors algorithms, Gaussian processes such as Gaussian process regression, cross-decomposition algorithms, including partial least squares and/or canonical correlation analysis; probabilistic graphical models including naive Bayes methods; models based on decision trees, such as decision tree classification algorithms. Additional machine-learning algorithms include ensemble methods such as bagging meta-estimator, AdaBoost, gradient tree boosting, and/or voting classifier methods. Details relating to various statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001), each of which is incorporated by reference. Additionally, ensemble techniques that combine different machine learning models can be used.

### C. Feature Selection

A 5-fold cross-validation procedure can be implemented for training the host classifier. In some instances, for each training fold, a lasso logistic regression was applied to training samples of the training fold for feature (gene) selection. The lasso regression can facilitate obtaining of a subset of predictors that minimize prediction error for a quantitative response variable (e.g., a probability value that identifies a likelihood of LRTI in a given subject). The lasso regression can impose a constraint on the host-classifier model parameters that causes regression coefficients for some variables to shrink toward zero.

As an illustrative example, the following genes can be selected from a first training fold: CCL22; EARS2; CISH; FN1; GNLY; IRF1; PTCD3; CCNA1; SLC38A2; RBP4; ANKRD22; KIAA1841; PCOLCE2; CXCL5; ZNF12; FABP4; ZNF708; FFAR3; AKR1C3; IARS1; ATP1A1-AS1; PSMB8; AC013457.1; CASC15; and SNURF.

With respect to lasso regression, a simple lasso logistic regression was fit using the cv.glmnet(family=‘binomial’) function from the R package glmnet, leaving all other parameters at their defaults. A 1se criterion was used for selecting the tuning parameter, which selects the sparsest value of the tuning parameter that lies within 1 standard error of the optimum. When evaluating test error, a tuning parameter was selected via nested cross-validation within the training set only.

### D. Training

The selected features from each of the training samples can be used to train the host classifier, and the host classifier can be applied to test samples in a corresponding test fold to obtain an out-of-fold host probability of having an LRTI. In some instances, six genes are selected using lasso logistic regression for determining a likelihood of LRI. For each test set, at least 9 No Evidence subjects were used to ensure sufficient negative samples in each test set.

The machine learning model may be trained until certain predetermined conditions for accuracy or performance are satisfied, such as having minimum desired values corresponding to diagnostic accuracy measures. For example, the diagnostic accuracy measure may correspond to prediction of a diagnosis or disease outcome in the subject. Examples of diagnostic accuracy measures may include sensitivity, specificity, positive predictive value (PPV), negative predictive value (NPV), accuracy, and area under the curve (AUC) of a Receiver Operating Characteristic (ROC) curve corresponding to the diagnostic accuracy of detecting or predicting LRTI.

### E. Classification Accuracy of the Host Classifier

As demonstrated below, the trained host classifier can perform accurate classification of LRTI across various biological samples.

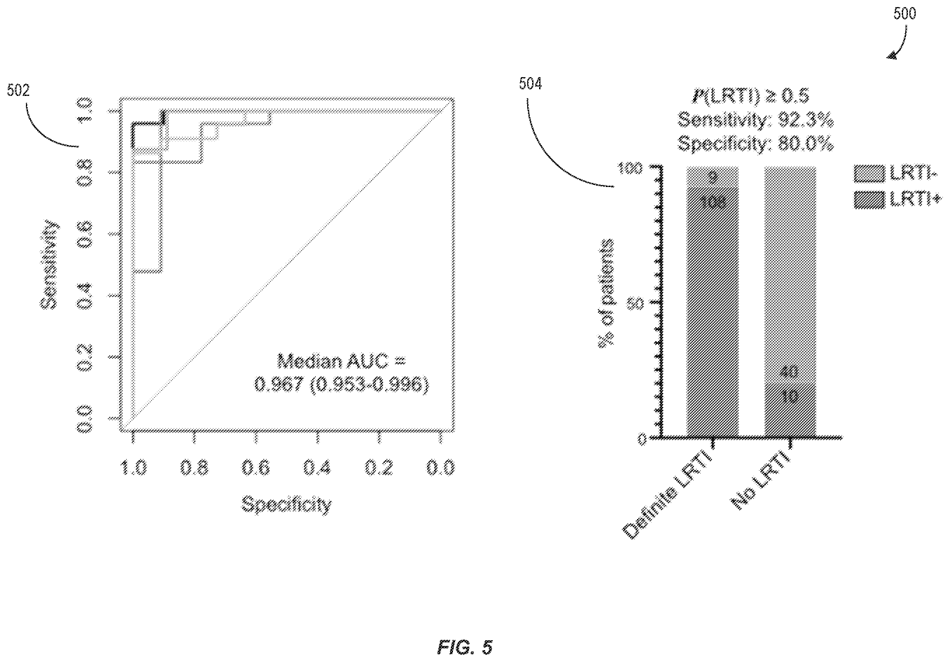

FIG. 5 shows a set of graphs 500 that identify classification accuracy of the trained classifier, according to some embodiments. In particular, FIG. 5 shows a Receiver operating characteristic (ROC) curve 502 showing the performance of the host gene classifier in each of the 5 cross-validation folds.

As shown in the ROC curve 502, the host classifier yielded a median AUC of 0.967 (range: 0.953-0.996). Table 2 shows the number of genes selected for use in the classifier ranging from 11 to 25 across the five train/test splits. Table 2 further shows that AUC associated with each training fold, which ranges from 0.953 to 0.996.

TABLE 2
Host genes selected by each of the 5 folds during cross-validation.

| Gene | Coefficient | Gene symbol | Fold | AUC |
|---|---|---|---|---|
| (Intercept) | −2.0280997 | NA | 1 | 0.996 |
| ENSG00000102962 | −0.0470626 | CCL22 | 1 | 0.996 |
| ENSG00000103356 | −0.005208 | EARS2 | 1 | 0.996 |
| ENSG00000114737 | 0.29736499 | CISH | 1 | 0.996 |
| ENSG00000115414 | −0.0963792 | FN1 | 1 | 0.996 |
| ENSG00000115523 | 0.40435424 | GNLY | 1 | 0.996 |
| ENSG00000125347 | 0.08788657 | IRF1 | 1 | 0.996 |
| ENSG00000132300 | −0.1094826 | PTCD3 | 1 | 0.996 |
| ENSG00000133101 | 0.13698008 | CCNA1 | 1 | 0.996 |
| ENSG00000134294 | 0.23225034 | SLC38A2 | 1 | 0.996 |
| ENSG00000138207 | −0.1867265 | RBP4 | 1 | 0.996 |
| ENSG00000152766 | 0.01618487 | ANKRD22 | 1 | 0.996 |
| ENSG00000162929 | −0.031873 | KIAA1841 | 1 | 0.996 |
| ENSG00000163710 | −0.0141204 | PCOLCE2 | 1 | 0.996 |
| ENSG00000163735 | −0.0202633 | CXCL5 | 1 | 0.996 |
| ENSG00000164631 | −0.0296153 | ZNF12 | 1 | 0.996 |
| ENSG00000170323 | −0.2260672 | FABP4 | 1 | 0.996 |
| ENSG00000182141 | −0.2407744 | ZNF708 | 1 | 0.996 |
| ENSG00000185897 | 0.2334893 | FFAR3 | 1 | 0.996 |
| ENSG00000196139 | −0.0145091 | AKR1C3 | 1 | 0.996 |
| ENSG00000196305 | −0.0541444 | IARS1 | 1 | 0.996 |
| ENSG00000203865 | −0.0785439 | ATP1A1-AS1 | 1 | 0.996 |
| ENSG00000204264 | 0.14737796 | PSMB8 | 1 | 0.996 |
| ENSG00000259094 | −0.0642868 | AC013457.1 | 1 | 0.996 |
| ENSG00000272168 | −0.0129449 | CASC15 | 1 | 0.996 |
| ENSG00000273173 | −0.1236364 | SNURF | 1 | 0.996 |
| (Intercept) | −0.4183313 | NA | 2 | 0.953 |
| ENSG00000115523 | 0.15221918 | GNLY | 2 | 0.953 |
| ENSG00000134294 | 0.21115084 | SLC38A2 | 2 | 0.953 |
| ENSG00000138207 | −0.2940594 | RBP4 | 2 | 0.953 |
| ENSG00000152766 | 0.06800369 | ANKRD22 | 2 | 0.953 |
| ENSG00000162929 | −0.0224986 | KIAA1841 | 2 | 0.953 |
| ENSG00000170323 | −0.1720699 | FABP4 | 2 | 0.953 |
| ENSG00000185897 | 0.17219249 | FFAR3 | 2 | 0.953 |
| ENSG00000187608 | 0.03953433 | ISG15 | 2 | 0.953 |
| ENSG00000196139 | −0.0991982 | AKR1C3 | 2 | 0.953 |
| ENSG00000204264 | 0.0974774 | PSMB8 | 2 | 0.953 |
| ENSG00000253729 | −0.2010562 | PRKDC | 2 | 0.953 |
| ENSG00000272821 | 0.05245633 | U62317.2 | 2 | 0.953 |
| (Intercept) | 0.27758103 | NA | 3 | 0.986 |
| ENSG00000008226 | −0.1276673 | DLEC1 | 3 | 0.986 |
| ENSG00000113068 | −0.0087115 | PFDN1 | 3 | 0.986 |
| ENSG00000114737 | 0.01066616 | CISH | 3 | 0.986 |
| ENSG00000115523 | 0.20275662 | GNLY | 3 | 0.986 |
| ENSG00000117143 | −0.0043278 | UAP1 | 3 | 0.986 |
| ENSG00000133101 | 0.22848885 | CCNA1 | 3 | 0.986 |
| ENSG00000134294 | 0.05523801 | SLC38A2 | 3 | 0.986 |
| ENSG00000136231 | 0.01793655 | IGF2BP3 | 3 | 0.986 |
| ENSG00000149021 | −0.0088169 | SCGB1A1 | 3 | 0.986 |
| ENSG00000163735 | −0.0087013 | CXCL5 | 3 | 0.986 |
| ENSG00000164631 | −0.0351792 | ZNF12 | 3 | 0.986 |
| ENSG00000170323 | −0.4076148 | FABP4 | 3 | 0.986 |
| ENSG00000170324 | −0.0607793 | FRMPD2 | 3 | 0.986 |
| ENSG00000185507 | 0.29760005 | IRF7 | 3 | 0.986 |
| ENSG00000185897 | 0.09319602 | FFAR3 | 3 | 0.986 |
| ENSG00000196139 | −0.0532949 | AKR1C3 | 3 | 0.986 |
| ENSG00000196189 | 0.03111383 | SEMA4A | 3 | 0.986 |
| ENSG00000232629 | −0.0158939 | HLA-DQB2 | 3 | 0.986 |
| ENSG00000259094 | −0.0637096 | AC013457.1 | 3 | 0.986 |
| ENSG00000272168 | −0.1499889 | CASC15 | 3 | 0.986 |
| ENSG00000272660 | −0.1024448 | AC090425.2 | 3 | 0.986 |
| ENSG00000272821 | 0.03959668 | U62317.2 | 3 | 0.986 |
| ENSG00000273173 | −0.0502438 | SNURF | 3 | 0.986 |
| (Intercept) | −5.181784 | NA | 4 | 0.954 |
| ENSG00000115523 | 0.23413097 | GNLY | 4 | 0.954 |
| ENSG00000133106 | 0.05899777 | EPSTI1 | 4 | 0.954 |
| ENSG00000135604 | 0.23226792 | STX11 | 4 | 0.954 |
| ENSG00000138207 | −0.1413799 | RBP4 | 4 | 0.954 |
| ENSG00000149021 | −0.0263191 | SCGB1A1 | 4 | 0.954 |
| ENSG00000158769 | −0.0055638 | F11R | 4 | 0.954 |
| ENSG00000163710 | −0.0206461 | PCOLCE2 | 4 | 0.954 |
| ENSG00000168394 | 0.13854581 | TAP1 | 4 | 0.954 |
| ENSG00000170323 | −0.2005039 | FABP4 | 4 | 0.954 |
| ENSG00000175073 | 0.50212823 | VCPIP1 | 4 | 0.954 |
| ENSG00000182141 | −0.1006603 | ZNF708 | 4 | 0.954 |
| ENSG00000185885 | 0.07866599 | IFITM1 | 4 | 0.954 |
| ENSG00000185897 | 0.13639215 | FFAR3 | 4 | 0.954 |
| (Intercept) | 0.5726104 | NA | 5 | 0.967 |
| ENSG00000115523 | 0.0762971 | GNLY | 5 | 0.967 |
| ENSG00000133106 | 0.02410902 | EPSTI1 | 5 | 0.967 |
| ENSG00000151914 | −0.029031 | DST | 5 | 0.967 |
| ENSG00000163710 | −0.0191247 | PCOLCE2 | 5 | 0.967 |
| ENSG00000163735 | −0.1149234 | CXCL5 | 5 | 0.967 |
| ENSG00000170323 | −0.389078 | FABP4 | 5 | 0.967 |
| ENSG00000175073 | 0.05579582 | VCPIP1 | 5 | 0.967 |
| ENSG00000185897 | 0.04184826 | FFAR3 | 5 | 0.967 |
| ENSG00000187608 | 0.00687972 | ISG15 | 5 | 0.967 |
| ENSG00000188820 | 0.03110363 | CALHM6 | 5 | 0.967 |
| ENSG00000204264 | 0.27278452 | PSMB8 | 5 | 0.967 |

FIG. 5 also shows a bar plot 504 showing the number and percentage of Definite and No Evidence samples that were classified according to their clinical adjudication using a 50% out-of-fold probability threshold. Using a 50% out-of-fold probability threshold to classify a patient as suffering from LRTI (LRTI+), the classifier assigned 92% of Definite patients and 80% of No Evidence patients according to their clinical LRTI adjudication. The results from FIG. 5 demonstrate that the host classifier can be trained using gene expression levels to accurately determine whether a given subjects has LRTI.

Having validated the performance of our approach by cross-validation, we then applied lasso logistic regression to all the Definite and No Evidence patients to select a final set of genes (n=14) for later classification of patients with Suspected or Indeterminate LRTI status.

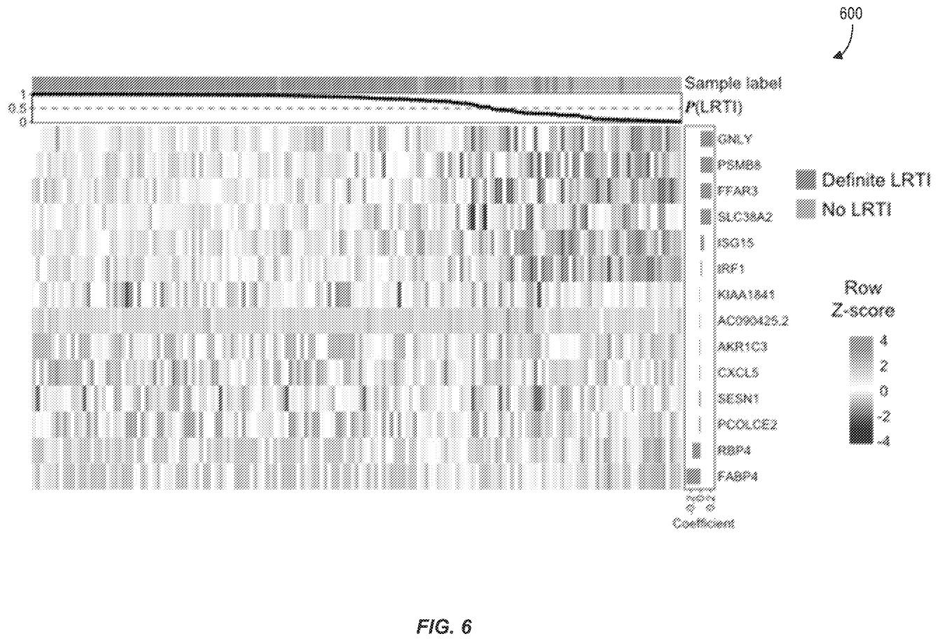

FIG. 6 shows a heatmap 600 showing the normalized expression across samples (columns) of the 14 final classifier genes (rows) selected when training on the complete Definite and No Evidence dataset. In addition to FIG. 6, Table 3 shows regression coefficients of each selected gene and the out-of-fold probability of LRTI assigned to each sample. Based on Table 3, the host classifier was trained using genes identified as having high absolute regression coefficients.

TABLE 3
Genes selected by the host classifier and their coefficients.

| | Gene | | Coeffi- |
|---|---|---|---|
| Gene ID | symbol | Gene product | cient |
| ENSG00000115523 | GNLY | granulysin | 0.257 |
| ENSG00000204264 | PSMB8 | proteasome subunit beta 8 | 0.249 |
| ENSG00000185897 | FFAR3 | free fatty acid receptor 3 | 0.224 |
| ENSG00000134294 | SLC38A2 | solute carrier family 38 | 0.214 |
| | | member 2 | |
| ENSG00000187608 | ISG15 | ISG15 ubiquitin-like | 0.070 |
| | | modifier | |
| ENSG00000125347 | IRF1 | interferon regulatory | 0.027 |
| | | factor 1 | |
| ENSG00000162929 | KIAA1841 | uncharacterized | −0.014 |
| ENSG00000272660 | AC090425.2 | uncharacterized | −0.016 |
| ENSG00000196139 | AKR1C3 | aldo-keto reductase family | −0.019 |
| | | 1 member C3 | |
| ENSG00000163735 | CXCL5 | C-X-C motif chemokine | −0.019 |
| | | ligand 5 | |
| ENSG00000080546 | SESN1 | sestrin 1 | −0.033 |
| ENSG00000163710 | PCOLCE2 | procollagen C-endopepti- | −0.033 |
| | | dase enhancer 2 | |
| ENSG00000138207 | RBP4 | retinol binding protein 4 | −0.167 |
| ENSG00000170323 | FABP4 | fatty acid binding protein 4 | −0.297 |

| | |
|---|---|
| (Intercept) | −3.112 |

As shown in FIG. 6 and Table 3, the genes with the most positive regression coefficients, corresponding to higher expression in the Definite group, included: (i) GNLY, encoding an anti-bacterial peptide present in cytolytic granules of cytotoxic T cells and natural killer cells; (ii) SLC38A2, encoding a glutamine transporter upregulated in CD28-stimulated T cells; (iii) FFAR3, encoding a G protein-coupled receptor activated by short-chain fatty acids that is induced by alveolar macrophages upon infection; and (iv) the interferon-stimulated genes PSMB8, ISG15 and IRF1.

In addition, the genes with the most negative regression coefficients, corresponding to lower expression in the Definite group, were: (i) FABP4, encoding a fatty acid-binding protein considered a marker of alveolar macrophages, whose expression in the lung decreases in patients with LRTI; and (ii) RBP4, encoding a retinol-binding protein, whose expression in the lung has also been shown to sharply decrease following onset of LRTI and whose expression by macrophages in vitro is depressed by inflammatory stimuli.

### F. Correlation Between Gene Expression and Subject Age

Then, the expression of the final classifier genes was examined as a function of patient age to confirm that the selection of genes was not influenced by the different age distributions of the Definite and No Evidence groups.

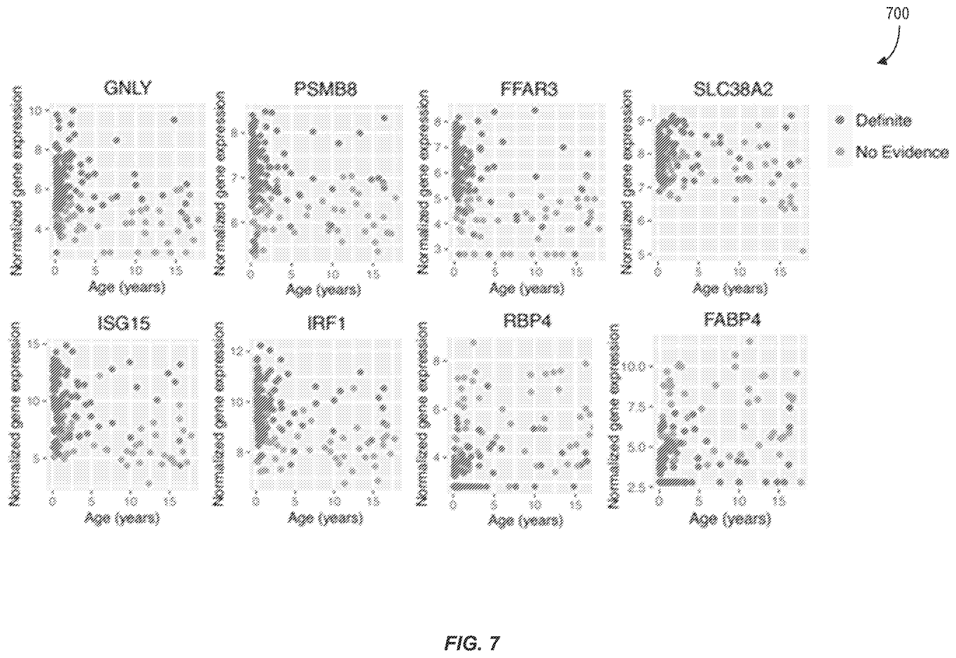

FIG. 7 shows an example set of graphs 700 that identify expression of the top eight host classifier genes by coefficient in LRTI^{definite} (red) and No-LRTI (blue) in subjects of different ages, according to some embodiments. The samples that were misclassified by the host gene classifier are highlighted (misclassified Definite patients in orange (n=9); misclassified No-LRTI patients in green (n=10)). Genes are shown in order of their coefficients in the host gene classifier. In FIG. 7, the six genes GNLY, PSMB8, FFAR3, SLC38A2, ISG15, and IFR1 were more highly expressed in LRTI^{definite} subjects across different ages. Conversely, RBP4 and FABP4 were more highly expressed in No-LRTI subjects across different ages. Based on the above results, the host classifier can be trained using expression levels of GNLY, PSMB8, FFAR3, SLC38A2, ISG15, and IFR1 to determine a classification of LRTI in pediatric subjects.

Further, Table 4 shows results of differential expression analysis by comparing No Evidence patients under four (n=23) versus over four years old (n=27). As shown in Table 4, there was no significant difference in the expression of the 14 genes when comparing No Evidence patients under the age of four (n=23) and over the age of four (n=27).

TABLE 4
Correlation between gene expression and age

| Gene | Log fold | | Adjusted |
|---|---|---|---|
| symbol | change | P value | P value |
| GNLY | −1.14 | 0.01 | 0.72 |
| PSMB8 | −0.33 | 0.21 | 0.82 |
| FFAR3 | −0.43 | 0.33 | 0.87 |
| SLC38A2 | −0.57 | 0.07 | 0.75 |
| ISG15 | −1.68 | 0.01 | 0.72 |
| IRF1 | −0.30 | 0.25 | 0.83 |
| KIAA1841 | 0.14 | 0.59 | 0.94 |
| AC090425.2 | 1.48 | 0.07 | 0.76 |
| AKR1C3 | 0.81 | 0.10 | 0.76 |
| CXCL5 | 0.24 | 0.67 | 0.95 |
| SESN1 | 0.27 | 0.47 | 0.91 |
| PCOLCE2 | 0.54 | 0.21 | 0.81 |
| RBP4 | −0.04 | 0.95 | 0.99 |
| FABP4 | −0.56 | 0.45 | 0.90 |

Table 5 additionally shows results of differential expression analysis comparing Definite (n=100) and No Evidence (n=23) patients under four years old. In Table 5, expression of 12 of the genes remained significantly different when comparing only children under the age of four in the Definite (n=100) and No Evidence (n=23) groups.

TABLE 5
Gene expression of subjects under 4 years old

| Gene | Log fold | | Adjusted |
|---|---|---|---|
| symbol | change | P value | P value |
| GNLY | 2.73 | 2.11E−08 | 1.46E−06 |
| PSMB8 | 1.11 | 7.78E−08 | 4.22E−06 |
| FFAR3 | 2.93 | 9.70E−12 | 4.25E−09 |
| SLC38A2 | 0.60 | 2.50E−05 | 4.33E−04 |
| ISG15 | 3.49 | 3.71E−09 | 3.89E−07 |
| IRF1 | 1.52 | 5.18E−12 | 2.88E−09 |
| KIAA1841 | −0.89 | 2.84E−05 | 4.76E−04 |
| AC090425.2 | −0.18 | 7.50E−01 | 8.56E−01 |
| AKR1C3 | −2.48 | 2.24E−12 | 1.42E−09 |
| CXCL5 | −2.63 | 4.62E−09 | 4.52E−07 |
| SESN1 | −0.53 | 4.92E−02 | 1.38E−01 |
| PCOLCE2 | −2.06 | 2.60E−09 | 2.93E−07 |
| RBP4 | −3.64 | 1.51E−17 | 1.00E−13 |
| FABP4 | −5.54 | 4.02E−26 | 5.36E−22 |

### G. Method

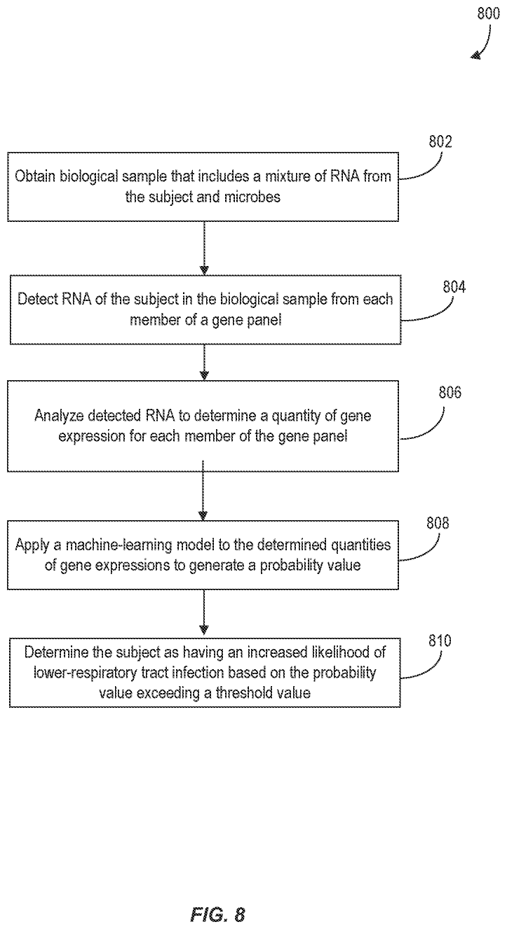

FIG. 8 is a flowchart for a method 800 for using machine-learning techniques to determine a likelihood of LRTI in a subject, according to some embodiments. The method 800 of can be used as one way to implement the host gene expression analysis described in the method 400 of FIG. 4. For example, a machine-learning model can be trained to determine a likelihood of LRTI in subjects based on expression levels for a set of genes known to be differentially expressed in LRTI subjects relative to control subjects. Once the machine-learning model is trained, only quantities of gene expression in the biological sample can be used to determine LRI, and the additional determination of differential gene expression using reference levels may be avoided. In some instances, the method 800 supplements or enhances the host gene expression analysis described in the method 400 of FIG. 4. Further, the machine-learning model can be trained and tested using gene expression of different subsets of the set of genes to identify a gene panel (e.g., Table 3) that provides the most accurate prediction of LRTI in subjects. Once the gene panel is identified, the method 400 of FIG. 4 can use the gene panel for determining a likelihood of LRTI in subjects. At least a portion of the method may be performed by a computer system.

At block 802, a biological sample of a subject is obtained. The biological sample can include a mixture of RNA from the subject and microbes. Exemplary biological samples are described herein and include those obtained, for example, by a nasal swab, nasopharyngeal swab, nasopharyngeal wash or aspirate, mid-turbinate nasal swab, oropharyngeal swab, buccal swab, a broncho-alveolar lavage, or an endotracheal aspirate. In some embodiments, the biological sample is serum, plasma, blood, or solid tissue. In some embodiments, a sample may be processed to provide or purify RNA of a particular nucleic acid molecule or fragment thereof.

At block 804, RNA of the subject in the biological sample from each member of a gene panel is detected. For example, RNA may be sequenced using sequencing methods such as next-generation sequencing, high-throughput sequencing, massively parallel sequencing, sequencing-by-synthesis, paired-end sequencing, single-molecule sequencing, nanopore sequencing, pyrosequencing, semiconductor sequencing, sequencing-by-ligation, sequencing-by-hybridization, RNA-Seq, Digital Gene Expression, Single Molecule Sequencing by Synthesis (SMSS), Clonal Single Molecule Array (Solexa), shotgun sequencing, Maxim-Gilbert sequencing, primer walking, and Sanger sequencing. Sequencing methods may comprise targeted sequencing, whole-genome sequencing (WGS), lowpass sequencing, bisulfite sequencing, whole-genome bisulfite sequencing (WGBS), or a combination thereof. Sequencing methods may include preparation of suitable libraries. Sequencing methods may include amplification of nucleic acids (e.g., by targeted or universal amplification, such as PCR). Gene expression may also be assessed by PCR, Loop-Mediated Isothermal Amplification (LAMP), Transcription-Mediated Amplification (TMA), Isothermal Amplification or other nucleic acid amplification assay.

In some instances, the gene panel includes at least two members from a group of genes identified in Table 3. For example, the gene panel can include at least two members selected from a group of genes consisting of GNLY, PSMB8, FFAR3, SLC38A2, ISG15, IRF1, KIAA1841, AC090425.2, AKR1C3, CXCL5, SESN1, PCOLCE2, RBP4, TAP1, EPSTI1, and FABP4. In other instances, the gene panel includes at least 3, 4, 5, 6, 7, 8, 9, 10, 11, 12, 13, 14, 15, or all of the genes of this group. The gene panel may thus comprise one or more genes set forth in the tables (e.g., Tables 1-3) and any additional genes identified as being correlated with LRTI risk. Differential gene expression of at least one gene of the above panel relative to reference levels can be indicative of a likelihood of LRTIs in subjects.

At block 806, the detected RNA is analyzed to determine a quantity of gene expression for each member of the gene panel. Gene expression levels may be determined using any suitable method. The quantity of the gene expression can include quantity and/or the frequency of RNA present in the biological sample for each member of the gene panel. In effect, each member of the gene panel (e.g., GNLY) can be associated with a corresponding quantity of gene expression, thereby obtaining respective quantities of gene expressions. The process for measuring gene expression levels is further described in Section VI of the present disclosure.

In some instances, the determined quantity can be a quantity of the differential gene expression relative to reference levels in control subjects. The steps for determining differential gene expression are further described in block 406 of FIG. 4.

At block 808, a machine-learning model is applied to the determined quantities of gene expressions to generate a probability value. The probability value can correspond to a predicted likelihood of the subject of having an LRTI. In some embodiments, the level of expression of each gene is weighted with a predefined coefficient. The predefined coefficients can be the same or different for the genes. The probability score can be determined in various ways, e.g., by statistical or machine learning regression or classification such as, but not limited to, linear regression, including least squares regression, ridge or LASSO regression, elastic net regression, regularized Cox regression, logistic regression, orthogonal matching pursuit models, a Bayesian regression model, or deep learning methods, such as convolutional neural networks, recurrent neural networks and generative adversarial networks (see, e.g., LeCun et al., Nature 521: 436-444, 2015).

The machine-learning model can be trained from a training set of samples obtained from confirmed LRTI patients (e.g. “Definite” subjects), e.g., determined by clinical adjudication and/or culture of organism from a blood or organ sample from a patient. The machine learning model may be trained until certain predetermined conditions for accuracy or performance are satisfied, such as having minimum desired values corresponding to diagnostic accuracy measures. For example, the diagnostic accuracy measure may correspond to prediction of a diagnosis or disease outcome in the subject. Examples of diagnostic accuracy measures may include sensitivity, specificity, positive predictive value (PPV), negative predictive value (NPV), accuracy, and area under the curve (AUC) of a Receiver Operating Characteristic (ROC) curve corresponding to the diagnostic accuracy of detecting LRTI.

In some instances, different subsets of genes are selected for training the machine-learning model (e.g., to determine the probability value) using all or a subset of the training samples (i.e., subjects for which LRTI status is known and for which expression of the genes was measured). This training subset can then be used to train (optimize) a model, whose accuracy can be measured, e.g., using the AUC of an ROC curve. Then, another subset of genes can be selected, with a further training process providing another model whose accuracy can also be measured. The accuracy can be measured using the training set or a validation set, which can include samples with known labels that were excluded from the training set. This process of generating models for different subsets of genes, along with the accuracy of each model, can continue, possibly for all possible subsets of genes for which expression levels have been measured. A panel providing the best accuracy can be selected, however the accuracy is measured.

At block 810, the subject is determined as having an increased likelihood of lower-respiratory tract infection based on the probability value exceeding a threshold value. As described in block 410 in FIG. 4, the threshold value can be selected based on a desired accuracy, e.g., a trade off of sensitivity and specificity. In some embodiments, likelihood of LRTI is assigned based on a cutoff value (also referred to as a threshold value) using a reference scale, e.g., from 0 to 1.0. In some embodiments, a cutoff value of 0.5 or greater may be employed to define likelihood of LRTI. A cutoff value of 0.5 can correspond to a probability of 50%. In some embodiments, LRTI likelihood may be further stratified, for example, likelihood of LRTI may be categorized as “high,” “intermediate,” or “low”, e.g., based on the highest tertile, intermediate tertile and bottom tertile.

### IV. Detecting LRT1 Pathogens in Subjects

In addition to the analysis of hots gene expression described in Sections II and III of the present disclosure, the microbial mNGS data obtained from the subject biological samples can be analyzed to nominate likely pathogens that can be considered as causing LRTI in subjects. For example, diversity indices (e.g., Shannon diversity index, Simpson diversity index) or abundance levels of microbial pathogens can be determined in biological samples to determine whether a subject likely has an LRTI. The microbial analysis can be used alone to accurately determine the LRTI classifications. In some instances, the features from the nominated pathogens are integrated into the LRTI classifier to increase confidence in the results. Further, the identity of the nominated pathogens can also be used to guide treatment.

A. mNGS and Background Filtering

To conduct microbial analysis for determining a likelihood of LRTI in subjects, TA samples of subjects were processed alongside water controls through the open-source CZ-ID (formerly called IDSeq) metagenomic analysis pipeline. The pipeline includes performing subtractive alignment of the human genome and then reference-based alignment of the remaining reads at both the nucleotide and amino acid level against sequences in the National Center for Biotechnology Information (NCBI) nucleotide (NT) and non-redundant (NR) databases, respectively. The alignment is followed by assembly of the reads matching each taxon. The processing of the TA samples can thus result in a count matrix of the microbial tax. Taxa with ≥5 read counts in the NT alignment and an average assembly nucleotide alignment ≥70 bp were retained for the microbial analysis.

Water controls can enable estimation of the number of background reads expected for each taxon. In some instances, the water controls are used to remove the contribution of contamination by microbes present in the laboratory environment or reagents. The estimation of background reads can be performed by modeling the number of background reads as a negative binomial distribution with mean and dispersion fitted on the negative controls. For each batch (sequencing run) and taxon, a mean parameter of the negative binomial distribution was estimated by averaging the read counts across all negative controls after normalizing by the total non-host reads. The estimated background reads were regularized by including the global average (across all runs) as an additional sample. A single dispersion parameter across all taxa and runs was estimated using the functions glm.nb( ) and theta.md( ) from the R package MASS. Taxa were then tested for whether they exceeded the count expected from the background distribution, and a Holm-Bonferroni correction was applied to all tests performed within the same patient sample. Taxa were considered present in a sample if they achieved an adjusted p-value<0.05.

### B. Detecting Viral Pathogens

Any virus with known ability to cause LRTI can be identified as a probable pathogen, based on an identification of remaining sequence reads after background filtering of a biological sample of the subject. In particular, viruses with known ability to cause LRTI that were present at an abundance statistically exceeding their background distribution were considered probable pathogens.

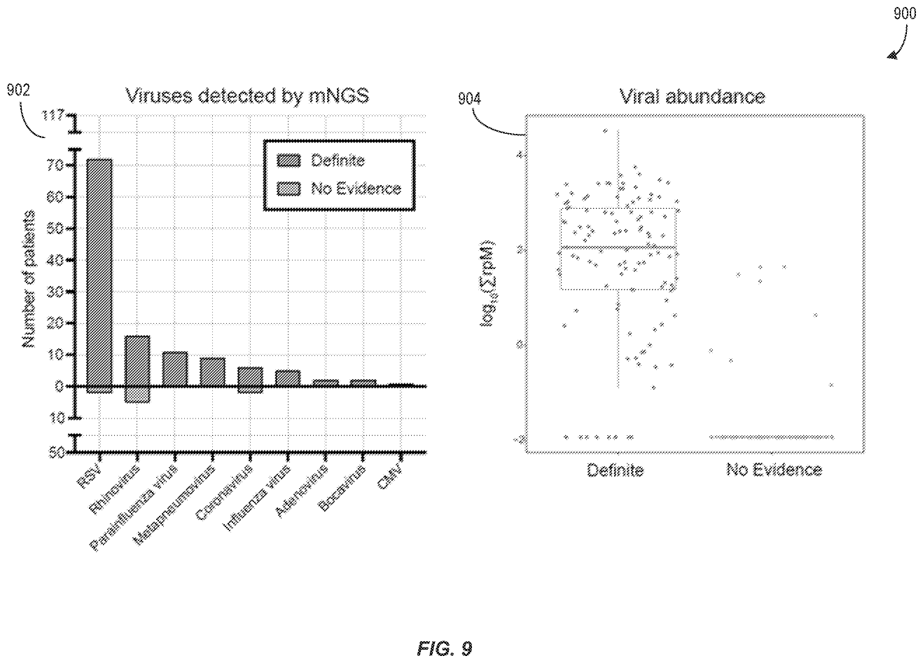

FIG. 9 shows a set of graphs 900 that identify abundance levels of viruses after background filtering, according to some embodiments. FIG. 9 shows a bar plot 902 showing the distribution of viruses detected by mNGS after background filtering in the Definite and No Evidence patients. The bar plot 902 shows that viruses were detected in the lungs of 107/117 (91%) Definite patients, with RSV the most prevalent. Among No Evidence patients, 8/50 (16%) also had viruses detected by mNGS, which were probably missed clinically in the absence of characteristic symptoms.

FIG. 9 shows a box plot 904 showing the summed abundance, measured in reads-per-million (rpM), of any pathogenic viruses detected in a patient, separated by group. The summed abundance is defined as a sum of all pathogenic viruses detected in a patient, measured in reads-per-million (rpM). The summed abundance (e.g., a direct sum or a weighted sum) can be used as the patient's “viral score” for later use in an integrated classifier. The box plot 904 shows that the abundance of viruses is significantly higher in Definite subjects compared to No Evidence subjects.

Because most Definite patients had a positive clinical Nnasopharyngeal (NP) swab viral PCR test, we could compare the viruses detected by PCR and mNGS. The comparison was complicated, however, by the fact that PCR was performed on upper airway samples. Accordingly, a virus detected by PCR was not necessarily causal of LRTI.

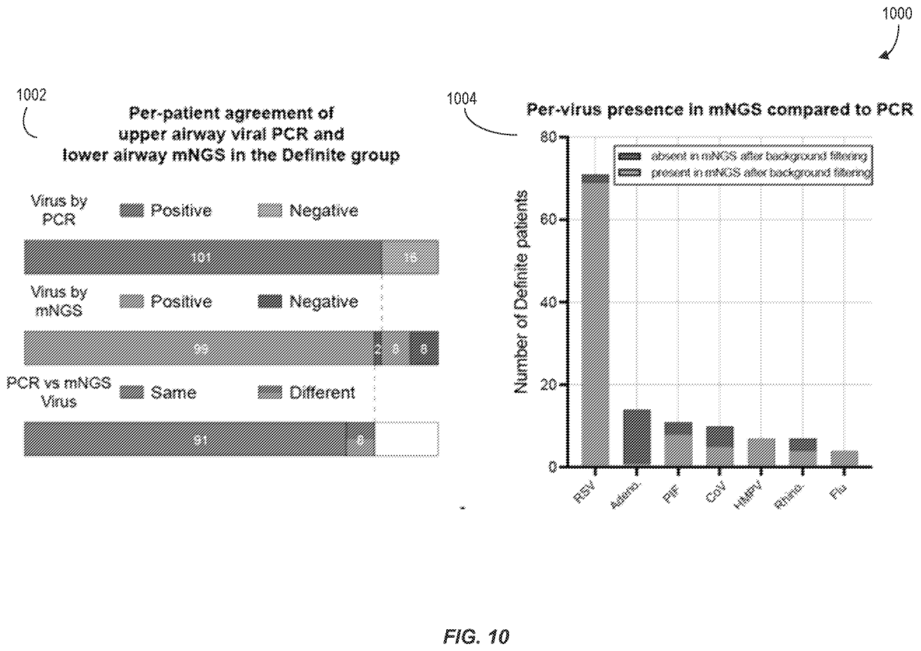

FIG. 10 shows a set of graphs 1000 that identify a comparison between using mNGS and PCR for detecting viruses in subjects diagnosed with LRTI, according to some embodiments. In particular, FIG. 10 shows a diagram 1002 depicting an agreement at the patient level between viral clinical testing and mNGS detection after background filtering in the Definite group. Agreement between the two methods in a patient was defined as at least one shared virus identified by both. The diagram 1002 showed that 99/101 (98%) Definite patients with a viral PCR hit also had a virus detected by mNGS, and both approaches detected at least one virus in common in 91 (92%) of those patients. However, mNGS alone detected viruses in 8/16 (50%) Definite patients lacking a viral PCR hit.

FIG. 10 also shows a bar plot 1004 showing the number of cases of each virus detected by upper respiratory PCR and the proportion that was also detected by mNGS after background filtering. In the bar plot 1004, viruses detected using PCR were also detected using mNGS, but for adenovirus.

To further validate the results shown in FIG. 10, Table 6 shows an agreement of mNGS TA viral detection with viral PCR results from NP swabs or from the same TA samples in a subset of Definite patients. As shown in Table 6, agreement reflects the number of viruses detected by mNGS out of the total number of viruses detected by PCR, and congruence reflects the number of viruses detected by both mNGS and PCR out of the total number of viruses detected by either method. In a subset of Definite patients where we performed viral PCR on the same TA samples subjected to mNGS (n=21), 96% of PCR hits were detected by mNGS. The congruence of PCR and mNGS in the same TA samples was higher than those of NP swabs. This may have been expected, as the NP swabs are obtained from a different part of the lung (e.g., upper airway samples).

TABLE 6
Agreement of mNGS and PCR

| | | Overall | | Overall |
|---|---|---|---|---|
| Patients with matched NP | Agreement of | congruence of | Agreement of | congruence of |
| and TA viral PCR testing | mNGS with NP | mNGS and NP | mNGS with TA | mNGS and TA |
| (n = 21) | swab PCR | swab PCR | PCR | PCR |
| All viruses | 22/34 = 64.7% | 22/37 = 59.5% | 23/24 = 95.8% | 23/26 = 88.5% |
| RSV | 11/12 = 91.7% | 11/12 = 91.7% | 10/10 = 100% | 10/11 = 90.9% |
| Rhinovirus | 4/7 = 57.1% | 4/10 = 40% | 6/6 = 100% | 6/7 = 85.7% |
| Adenovirus | 0/6 = 0% | 0/6 = 0% | 0/0 = 100% | 0/0 = 100% |
| Coronavirus | 2/3 = 66.7% | 2/3 = 66.7% | 2/2 = 100% | 2/2 = 100% |
| Human metapneumovirus | 3/3 = 100% | 3/3 = 100% | 3/3 = 100% | 3/3 = 100% |
| Parainfluenza virus | 2/3 = 66.7% | 2/3 = 66.7% | 2/3 = 66.7% | 2/3 = 66.7% |

### C. Diversity Model for Detecting Microbial and Fungal Pathogens

During an infection, it has been found that few pathogens would crowd out other microbes that would normally be present in a biological sample. This could be attributed to those few pathogens multiplying disproportionately during the infection. As a result of the increase of select pathogens, a loss of diversity of the respiratory microbiome can occur. Thus, for bacteria and fungi that were present after background filtering, a rules-based algorithm (the “diversity model”) for distinguishing potential pathogens from likely commensals based on their respective abundance levels. The diversity model can be effective for pediatric patients, as asymptomatic carriage of pathogenic bacteria is common in children. The diversity model thus identifies bacteria and fungi with known pathogenic potential that are relatively dominant in a sample, based on the principle that uncontrolled growth of a pathogen leads to reduced lung microbiome alpha diversity in the context of LRTI.

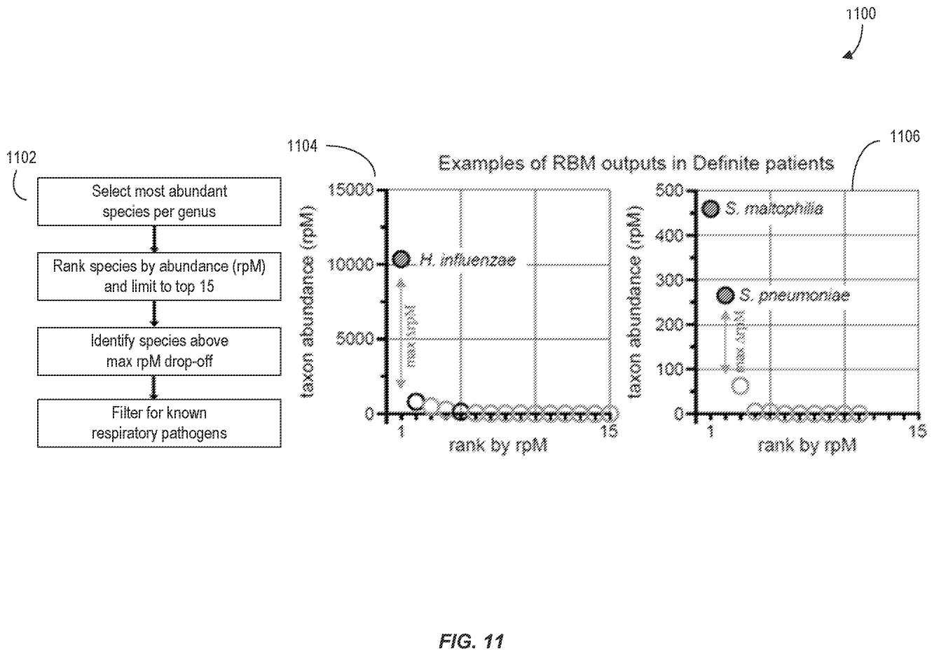

FIG. 11 illustrates example processes 1100 for determining potential pathogens that contribute to LRTI in subjects, according to some embodiments. A flowchart 1102 shows a process of a diversity model designed to identify potential bacterial/fungal pathogens in the context of LRTI. Initially, the remaining RNA after background filtering can be compared with bacterial and fungi genomes to identify types of species with which the remaining RNA are associated. We retained only the most abundant bacterial/fungal species from each genus. In case a less abundant species in the genus had known ability to cause LRTI, we retained it as well. We then ranked these species from greatest to least overall abundance (rpM) in the sample and retained the top 15 bacterial species. In some instances, the number of retained species are less than 15. The largest drop (e.g., a “gap threshold”) in abundance between the ranked species in the sample can be identified. All species with an abundance above the largest drop were selected. In effect, few microbe species that were disproportionately abundant to other species can be identified. If any of the species above the largest drop had a known ability to cause LRTI, such species was identified as a potential pathogen by the diversity model. For example, the species above the gap threshold were compared with a curated list of 50 or so established respiratory pathogens from five different landmark epidemiology studies, to filter out any microbes that are commensals or those that may be unknown for causing LRTI in subjects.

Graphs 1104 and 1106 show graphical illustrations of the results generated by the diversity model for two Definite patients. Each dot represents a bacterial/fungal species most abundant in its respective genus. A species on the list of known respiratory pathogens has a black outline, otherwise the outline is gray. A species above the maximum drop-off in rpM has a red fill, otherwise the fill is white. In the graph 1104, the results of the diversity model indicate H. influenzae as a potential pathogen that causes LRTI for a first subject. In the graph 1106, the results of the diversity model indicate S. maltophilia and S. pneumoniae as potential pathogens that cause LRTI for a second subject. Depending on the species of the pathogens, a different treatment can be selected thereby significantly improving the chances of recovery for a given subject.

### 1. Identification of Respiratory Pathogens Using the Diversity Model

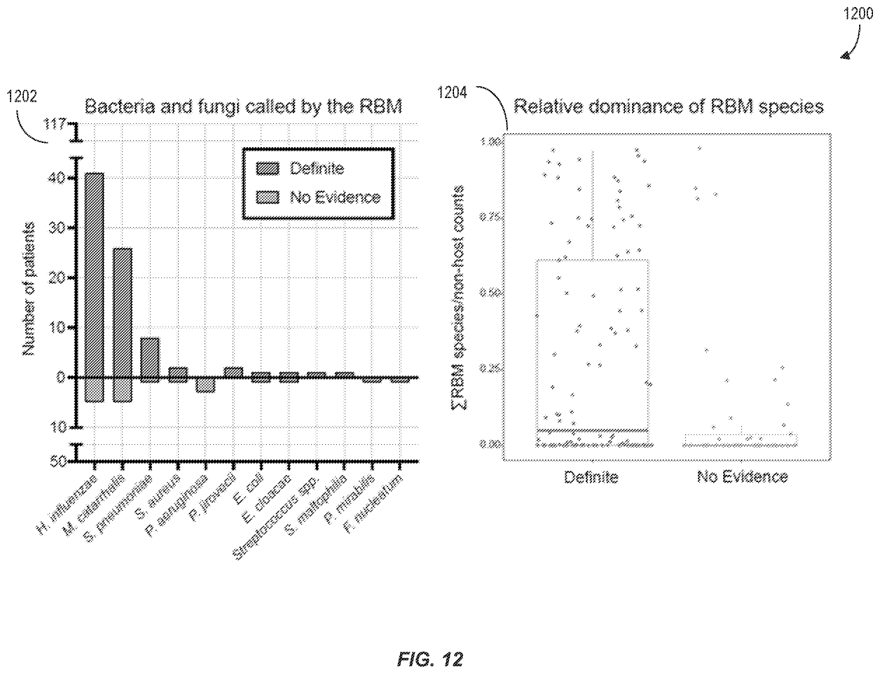

FIG. 12 shows a set of graphs 1200 that identify different characteristics of microbes detected by using the diversity model, according to some embodiments. For example, a bar plot 1202 shows a distribution of bacteria/fungi called as potential pathogens by the diversity model in the Definite and No Evidence patients. As shown in the boxplots 1204, the diversity model identified possible bacterial/fungal pathogens in 78/117 (66%) Definite patients, with the most common being H. influenzae, Moraxella catarrhalis and Streptococcus pneumoniae. The diversity model identified potential bacterial/fungal pathogens in 17/50 (34%) No Evidence patients.

In another example, the boxplots 1204 show the proportion of the identified pathogens out of all non-host counts in each subject, separated by Definite and No Evidence groups. The identified species was far less dominant in No Evidence groups relative to the dominance of identified specifies in Definite groups. Based on these differences, the proportion of the identified pathogens out of the non-host counts can be used as a “bacterial score” for determining a measure of relative dominance of pathogen species. The bacterial score can be used as input to other classifiers, such as an integrated classifier described in Section V of the present disclosure.

We next sought to compare the bacterial and fungal pathogens identified by mNGS with those found by culture of TA samples. The comparison can facilitate a determination of whether mNGS can be used as an alternative to culture tests, as mNGS can detect organisms that are challenging to grow in culture or are inhibited by previous antibiotic treatment. In addition, the diversity model identifies the likeliest pathogen based on a global view of the microbiome.

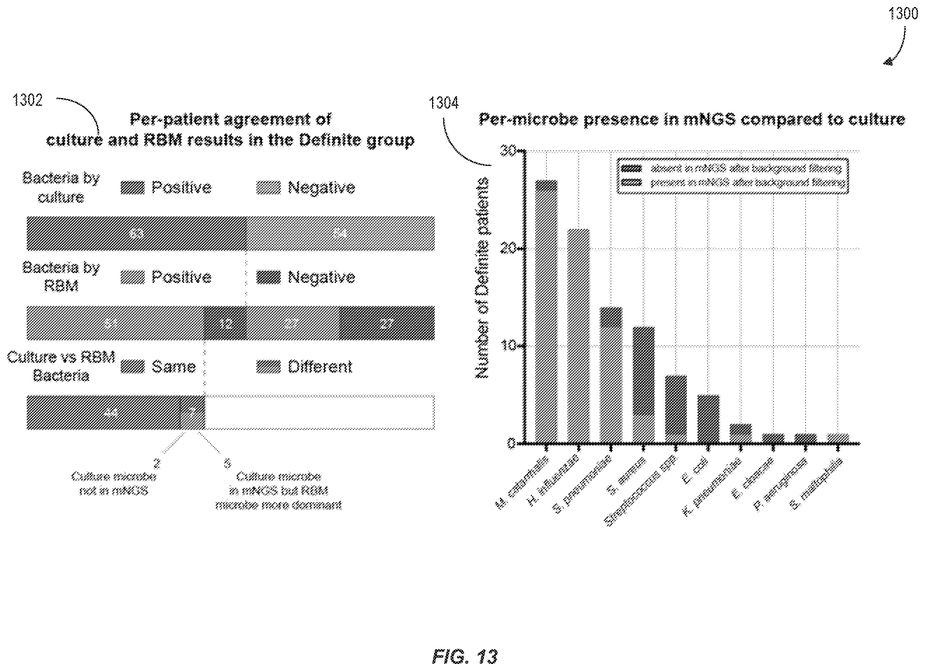

FIG. 13 shows a set of graphs 1300 that identify a comparison between using mNGS and culture tests for detecting bacterial pathogens in subjects diagnosed with LRTI, according to some embodiments. FIG. 13 shows a diagram 1302 depicting an agreement at the patient level between clinical culture and the results of the diversity model in the Definite group. Agreement between the two methods in a patient was defined as at least one shared species identified by both. As shown in the diagram 1302, we found that in 44/63 (70%) Definite subjects who had a positive culture, at least one pathogen identified by the diversity model was also found by culture. In the remaining 19 of 63 patients, the diversity model identified a different species than culture (n=7), or no pathogen at all (n=12). Even in these cases, the species grown in culture was usually present in the mNGS data, but the diversity model showed other species being more dominant. In addition, the diversity model was able to identify a potential pathogen in 27/54 (50%) Definite patients that lacked a positive culture.

FIG. 13 also shows a bar plot 1304 showing the number of cases of each species detected by culture and the proportion that was also detected by mNGS after background filtering. Most cases where the species grown in culture was absent from the mNGS data after background filtering involved Staphylococcus aureus, Streptococcus species. The exception to the similarities between mNGS data and culture tests included S. pneumoniae and Escherichia co/i.

### 2. Microbial Diversity Index

As described above, the lack of microbial diversity in a given biological sample can be predictive of an infection (e.g., LRTI) in subjects. To leverage such findings, a microbial diversity index (e.g., Shannon diversity index, Simpson diversity index) was calculated using either all viral and bacterial taxa, or only bacterial taxa, that were present after background filtering using the R package Vegan. The diversity index can correspond to an indicia of alpha diversity of microbes for a given biological sample. A low microbial diversity index can indicate that subject likely has an LRTI, relative to other subjects with high microbial diversity index. The determination of the diversity index allows to determine whether a given sample includes: (i) a single or a few dominant microbes; or (ii) multiple microbes that are present at relatively equal abundances. If the sample includes few dominant microbes being disproportionately represented compared to other microbes, the corresponding diversity index would have a lower value. Conversely, the diversity index would have a high value if many different microbes at similar abundance levels are found.

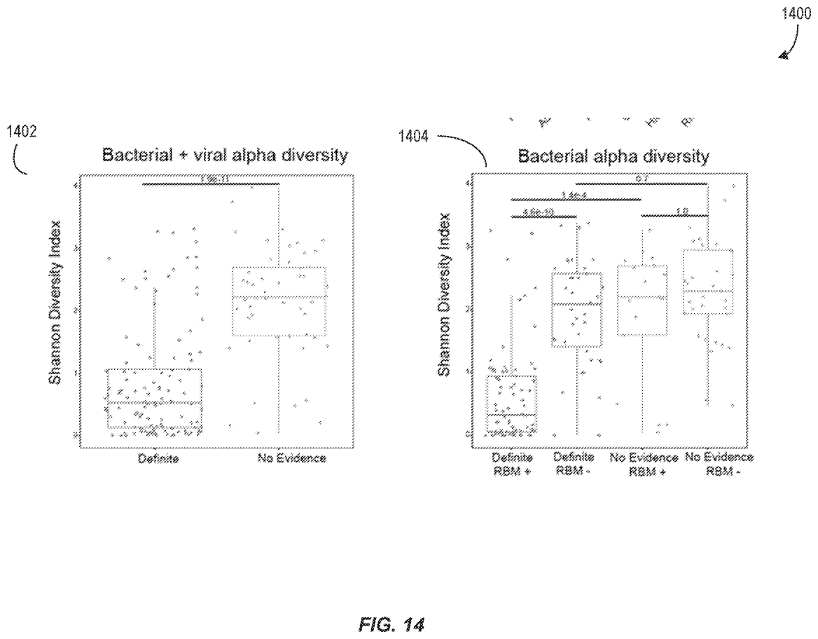

FIG. 14 shows a set of boxplots 1400 that show a correlation between microbial diversity and occurrence of LRTIs in subjects, according to some embodiments. To generate the boxplots, bacterial and fungal taxa in the mNGS data also underwent background filtering to retain only those present at an abundance statistically exceeding their background distribution based on water controls. Boxplots 1402 show bacterial+viral microbiome alpha diversity measured by the Shannon index in Definite and No Evidence patients. The boxplots 1402 show that diversity indices for Definite patients are lower relative to the diversity indices of No Evidence patients. A threshold for determining an increased likelihood for infection can be determined based on the separation of these two groups (cohorts). A threshold can be selected based on a tradeoff of sensitivity and specificity, e.g., a diversity index of about 1.3 could be used based on the data in boxplots 1402. Thus, the threshold can be determined based on one or more reference subjects having a known classification of whether a lower-respiratory tract infection exists. In the example of FIG. 14, the classifications are Definite and No Evidence.

Boxplots 1404 show bacterial-only alpha diversity measured by the Shannon index. Definite and No Evidence patients were further divided by whether a potential pathogen was identified by the diversity model. The P-values in the boxplots 1402 and 1404 were calculated by a Mann-Whitney test with Bonferroni correction. Mann-Whitney tests with Bonferroni correction were used to evaluate statistical significance of group differences. Similar to the boxplots 1402, the boxplots 1404 show that diversity indices for Definite patients are lower relative to the diversity indices of No Evidence patients. Further, subjects in the Definite group with pathogen identified by the diversity model exhibited markedly lower bacterial alpha diversity compared to Definite patients without pathogen identified by the diversity model and to No Evidence subjects. Nevertheless, No Evidence patients for which the identified pathogens were identified by the diversity model did not exhibit a noticeable loss of bacterial alpha diversity compared to No Evidence patients for which the pathogens were not identified by the diversity model.

As with boxplots 1402, a threshold for classifying a subject as having an increased likelihood can be determined based on the separation of these two groups (cohorts). More than one threshold may be used in any of the embodiments described herein. For example, different thresholds can correspond to different likelihood values, with increasing or decreasing likelihoods depending on how a parameter is defined. Using the Shannon diversity index (e.g., as shown in FIG. 14), a lower parameter value can indicate a higher likelihood of having the infection.

### D. Gene Expression Between Viral and Bacterial LRTI

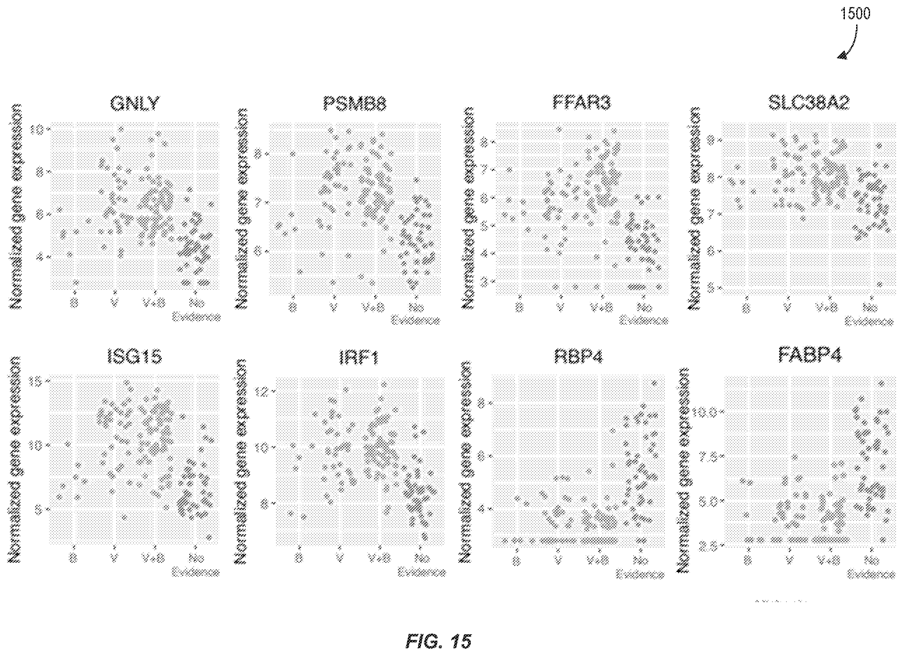

FIG. 15 shows a set of graphs 1500 that show a correlation between gene expression in subjects with underlying type of infections, according to some embodiments. In particular, FIG. 15 shows expression of eight genes identified in Definite subjects and No Evidence subjects (n=50), in which the Definite subjects were further divided into a first group in which only bacterial pathogens were called by the diversity model (n=7), a second group in which only viral pathogens detected by mNGS (N=36), a third group in which bacterial and viral pathogens were both detected (n=71). Three subjects from the Definite group are not shown because they did not have any pathogens identified by mNGS. Only one No Evidence sample in the plot of SLC38A2 was omitted since it was an extreme outlier. In the set of graphs 1500, “B” indicates bacterial infection, “V” indicates viral infection, and “V+B”=viral+bacterial co-infection.

As shown in FIG. 15, mNGS was able to identify viral and/or bacterial pathogens in 114/117 (97%) Definite patients. Having established by mNGS which Definite patients had an exclusively bacterial infection (n=7), an exclusively viral infection (n=36), or a viral/bacterial co-infection (n=71), it was examined how effectively the top host classifier genes captured these different scenarios. As expected, some of the interferon-stimulated genes (e.g., ISG15) provided much more discriminating power for Definite patients with a viral infection as compared to those with a purely bacterial infection. However, most other classifier genes appeared to behave similarly regardless of the underlying infection type.

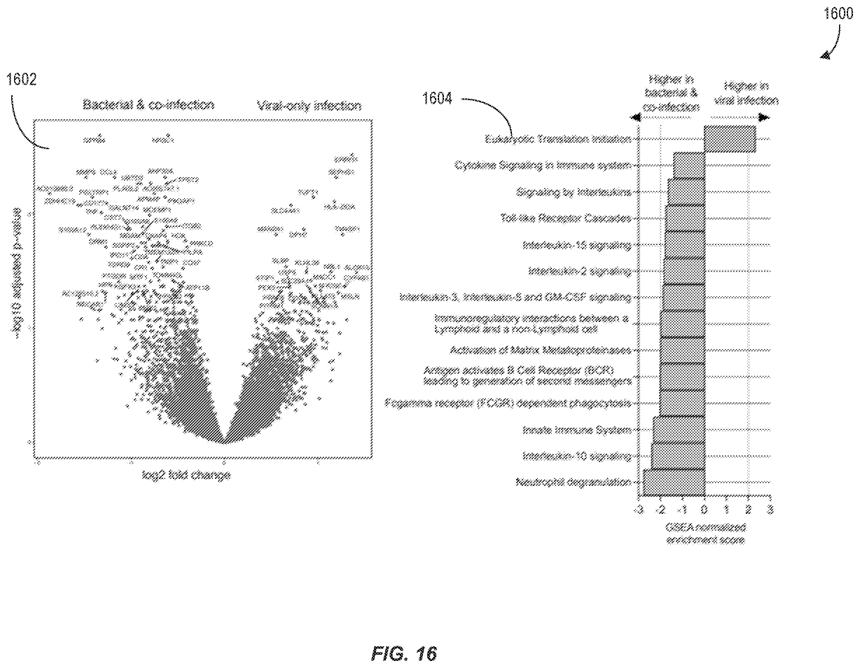

FIG. 16 shows a set of graphs 1600 that identify the difference of gene expression levels between co-infection samples and virus-only infection samples, according to some embodiments. In FIG. 16, a volcano plot 1602 show genes differentially expressed (DE) between Definite patients with any bacterial infection (bacterial-only+co-infection) and viral-only infection. Genes colored in purple reached statistical significance (adjusted p-value<0.05). We then asked more broadly whether host gene expression differed between patients with any bacterial LRTI (including viral co-infection) and patients with purely viral LRTI. Based on the volcano plot 1602, 108 differentially expressed genes (e.g., CCL4) were identified at an adjusted p-value<0.05.

FIG. 16 further shows normalized enrichment scores 1604 of selected REACTOME pathways that reached statistical significance (adjusted p-value<0.05) in the GSEA using the DE genes. It was also found that genes related to neutrophil degranulation and cytokine signaling were enriched in patients with any bacterial LRTI. These results suggest the potential for selecting appropriate treatment for the subjects depending on an underlying type of infection present in the corresponding biological sample.

### E. Method

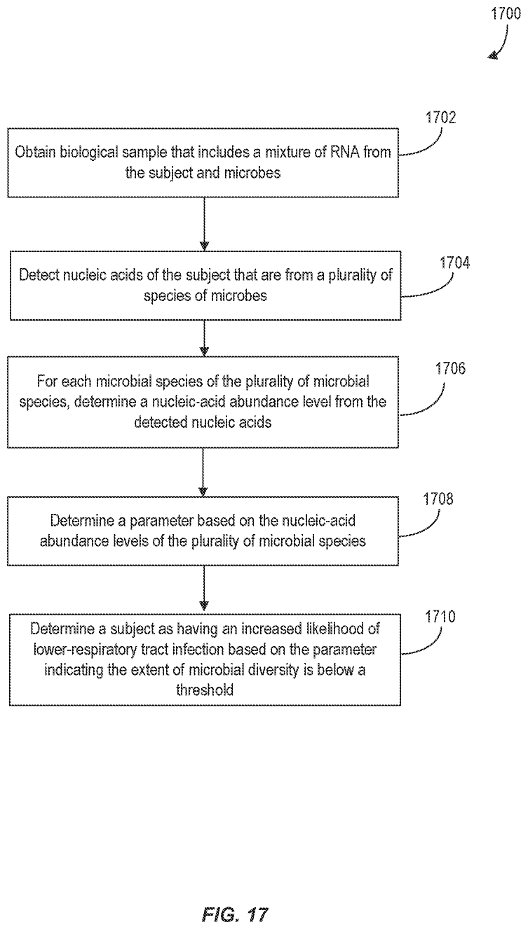

FIG. 17 is a flowchart for a method 1700 for using pathogen abundance levels to determine a likelihood of LRTI in a subject, according to some embodiments. At least a portion of the method may be performed by a computer system.

At block 1702, a biological sample of a subject is obtained. The biological sample can include a mixture of nucleic acids from the subject and microbes. The nucleic acids can include DNA and/or RNA. Exemplary biological samples are described herein and include those obtained, for example, by a nasal swab, nasopharyngeal swab, nasopharyngeal wash or aspirate, mid-turbinate nasal swab, oropharyngeal swab, buccal swab, a broncho-alveolar lavage, or an endotracheal aspirate. In some embodiments, the biological sample is serum, plasma, blood, or solid tissue. In some embodiments, a sample may be processed to provide or purify nucleic acids of a particular nucleic acid molecule or fragment thereof.

At block 1704, nucleic acids of the subject in the biological sample are detected. In some instances, each nucleic acid is from a particular species of microbes of a plurality of species of microbes. To determine the nucleic acids from the species of microbes, nucleic acids from the subject can filtered out (e.g., sequences that align to a human reference genome). For example, the sequence reads of the biological sample can be aligned to the human reference genome. A subset of sequence reads aligning to the human reference genome can be filtered out. The remaining sequence reads can then be realigned to the one or more reference microbe genomes. A sequence read of the remaining sequence reads that aligns to a reference microbe genome of a particular microbe species (e.g., B. fragilis) can be determined the sequence read as being associated the particular microbe species.

For example, nucleic acids may be sequenced using sequencing methods such as next-generation sequencing, high-throughput sequencing, massively parallel sequencing, sequencing-by-synthesis, paired-end sequencing, single-molecule sequencing, nanopore sequencing, pyrosequencing, semiconductor sequencing, sequencing-by-ligation, sequencing-by-hybridization, RNA-Seq, Digital Gene Expression, Single Molecule Sequencing by Synthesis (SMSS), Clonal Single Molecule Array (Solexa), shotgun sequencing, Maxim-Gilbert sequencing, primer walking, and Sanger sequencing. Sequencing methods may comprise targeted sequencing, whole-genome sequencing (WGS), lowpass sequencing, bisulfite sequencing, whole-genome bisulfite sequencing (WGBS), or a combination thereof. Sequencing methods may include preparation of suitable libraries. Sequencing methods may include amplification of nucleic acids (e.g., by targeted or universal amplification, such as PCR). Gene expression may also be assessed by PCR, Loop-Mediated Isothermal Amplification (LAMP), Transcription-Mediated Amplification (TMA), Isothermal Amplification or other nucleic acid amplification assay.

At block 1706, for each microbial species of the plurality of microbial species, a nucleic-acid abundance level from the detected nucleic acids is determined. In some instances, abundance levels of microbial species are determined by determining the number of sequence reads (e.g., rpM) that are mapped to individual species of microbes.

At block 1708, a parameter is determined based on the nucleic-acid abundance levels of the plurality of microbial species. The parameter can be indicative of an extent of microbial diversity in the biological sample. In some instances, the parameter corresponds to a statistical value (e.g., a weighted sum) of abundance levels of a set of microbe species, in which each species the set of microbe species has an abundance level above a gap threshold. To determine the gap threshold, for each genus of microbes, the species of the genus having the highest abundance level is selected. Then, the selected species are ranked by abundance level in sequential order, typically from highest to lowest. Based on the sequential order, the gap threshold can then be determined, in which the gap threshold can correspond to the abundance level at which the greatest difference in abundance level occurs between sequential microbes. For example, with the ranking being from highest to lowest, the highest abundance level may differ by 4.5 (e.g., 8-3.5) from the second highest, which might differ by only 0.8 from the third highest. The further differences (gaps) between other rankings can be even less. Thus, the gap threshold can be any value between 8 and 3.5, so that only the highest abundance would qualify. In another scenario, the largest gap can be between the second highest and the third highest, e.g., with the set of abundance values being 9, 8, 2, 1.5, 1, . . . . The gap threshold could be any abundance between 8 and 2. Additionally or alternatively, each species of the set of microbe species can be determined based on whether it is a known pathogen for causing LRTI. In this manner, the set of microbe species can correspond to known pathogens that have abundance levels above the gap threshold.

In some instances, the parameter corresponds to is a diversity index (e.g., Shannon diversity index). The diversity index can correspond to an indicia of alpha diversity of microbes for a given biological sample. A low microbial diversity index can indicate that subject likely has an LRTI, relative to other subjects with high microbial diversity index. The diversity index is generated at least by: normalizing, for each microbial species of the plurality of microbial species, nucleic-acid abundance level of the microbial species; and determining a negative sum of the normalized nucleic-acid abundance levels.

At block 1710, the subject is determined as having an increased likelihood of lower-respiratory tract infection based on the parameter indicating the extent of microbial diversity is below a threshold. The threshold can be selected based on a desired accuracy, e.g., a trade off of sensitivity and specificity. In some embodiments, likelihood of LRTI is assigned based on a cutoff value using a reference scale, e.g., from 0 to 1.0. In some embodiments, a cutoff value of 0.5 or greater may be employed to define likelihood of LRTI. In some embodiments, LRTI likelihood may be further stratified, for example, likelihood of LRTI may be categorized as “high,” “intermediate,” or “low”, e.g., based on the highest tertile, intermediate tertile and bottom tertile. In some embodiments, the threshold can be determined based on one or more reference subjects having a known classification of whether a lower-respiratory tract infection exists.

### V. Determining a Classification of LRT1 Using an Integrated Classifier

The host classifier and the microbial analyses (e.g., viral abundance, the diversity model) can individually predict LRTIs in subjects with sufficient accuracy. Moreover, the host classifier and the microbial analyses can be combined into an integrated classifier to further enhance the performance of classifying whether a subject has an LRTI. For example, the probability output from the host classifier can be combined with a score that is derived from the sum of the relative abundance of viral calls within a patient and the relative abundance of bacterial or fungal pathogens called by the diversity model. The combination of three scores can be incorporated into a logistic regression model to generate the integrated classifier that takes into account not only the host response to LRTIs but also these microbial features involved in causation of LRTIs.

### A. Schematics of the Integrated Classifier

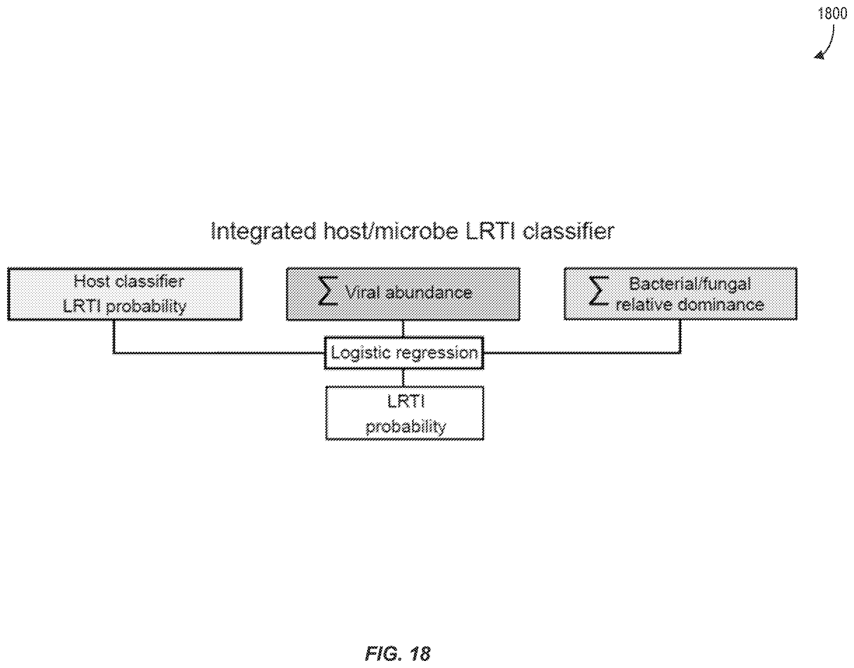

FIG. 18 shows a schematic diagram 1800 for using an integrated classifier to determine a likelihood of LRTI in subjects, according to some embodiments. As shown in the schematic diagram 1800, the schematic of the integrated classifier can include incorporating three input features into a logistic regression model in order to calculate the probability of LRTI. For example, we fit a logistic regression model on the following features: (i) the probability value generated by the host classifier as described in Section III of the present disclosure; (ii) the summed abundance, measured in reads-per-million (rpM), of any pathogenic viruses present after background filtering (“viral score”) as described in Section IV.B of the present disclosure; and (iii) the proportion of any potentially pathogenic bacteria/fungi identified by the diversity model out of all non-host read counts (“bacterial score”) as described in Section IV.C of the present disclosure. The processing of the three features using the logistic regression model generates a probability value indicative of whether a given subject has an LRTI.

### B. Evaluation of the Integrated Classifier

To determine whether integrating the host and microbial features could improve the performance of metagenomic LRTI classification, the integrated classifier was evaluated using 5-fold cross-validation using the same train/test splits from the host classifier cross-validation and the same per-fold host classifiers. To avoid any leakage from the test set affecting the host probabilities, we used the out-of-bag random forest votes as the host probabilities of the training samples.

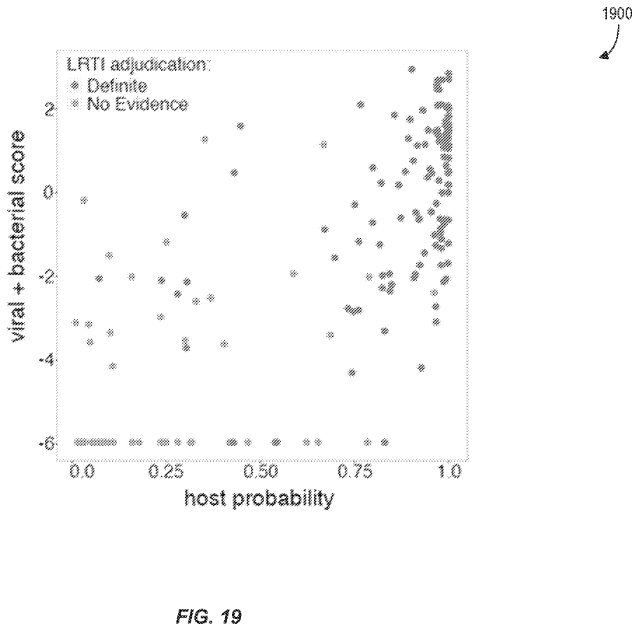

Before fitting the integrated classifier, we applied a logistic (log-odds) transformation to the host probabilities. In order to apply this transformation, we slightly regularized the raw probabilities. For the viral/bacterial scores, we applied a log₁₀ transformation. In order to avoid taking the log of 0, we added a small uniform quantity to the scores of all the samples, which was calculated by taking the minimum non-zero viral or bacterial score, respectively, in the training set and dividing it by 10. FIG. 19 shows a scatterplot 1900 of the host LRTI probability (x-axis) and the sum of the log₁₀-transformed microbial features (y-axis) in the Definite and No Evidence patients. As expected, the host features and microbial features were correlated across most samples as shown by the clusters of red dots on the right part of the scatterplot 1900, although there was some discordance between the above features.

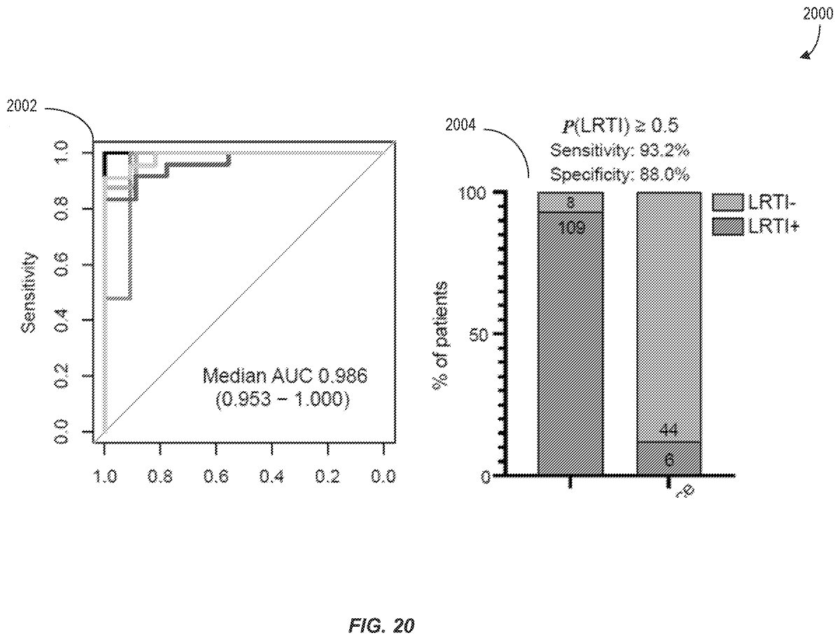

FIG. 20 shows a set of graphs 2000 that identify evaluation results of the integrated classifier, according to some embodiments. FIG. 20 depicts an ROC curve 2002 showing the performance of the integrated classifier on Definite and No Evidence patients across the 5 cross-validation folds. The integrated classifier achieved an AUC of 0.986 (range: 0.953-1.000) when assessed by 5-fold cross-validation, applying the same train/test splits used in the host-only cross-validation. As shown in the ROC curve 2002, the classification accuracy of the integrated classifier is greater than using the host classifier alone (e.g., AUC of 0.963 as shown in the ROC curve 502 in FIG. 5).

FIG. 20 also depicts a bar plot 2004 showing the number and percentage of Definite and No Evidence patients that were classified according to their clinical adjudication using a 50% out-of-fold probability threshold. Based on the bar plot 2004, the integrated classifier assigned 109/117 (93%) Definite patients as LRTI+ and 44/50 (88%) No Evidence patients as LRTI-. Thus, the integrated classifier demonstrated a high level of sensitivity (92.3%) and specificity (80.0%), thus identifying its potential for determining a likelihood of a subject having an LRTI.

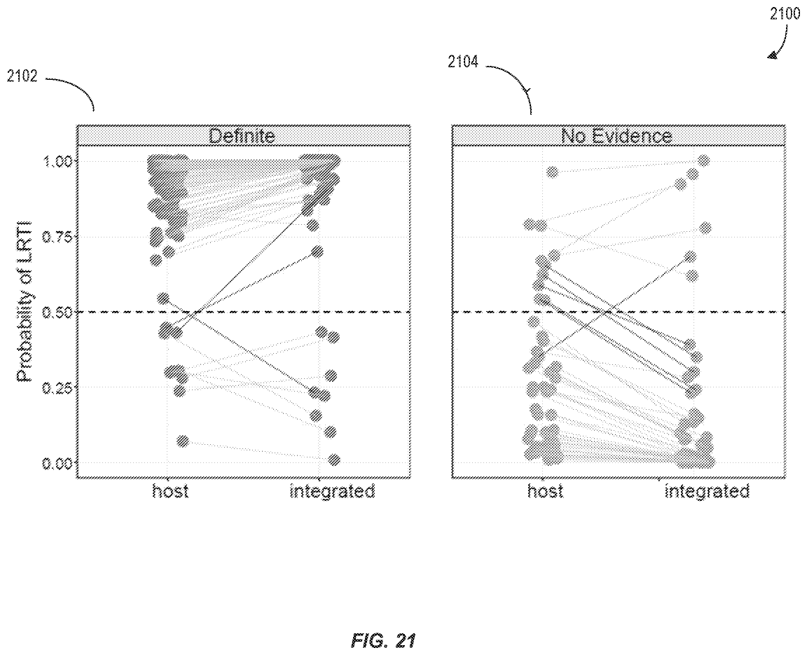

In addition, FIG. 21 shows comparison data 2100 between the probability of LRTI derived from the host classifier and the integrated classifier for Definite (left panel) 2102 and No Evidence (right panel) 2104 subjects. Dark connecting lines represent samples which switched their classification between the two classifiers.

Compared to the host-only classifier, the integrated classifier correctly identified a net of five additional patients according to their clinical adjudication. In the comparison data 2100, the probability of lower respiratory infection can range from zero to one, at which outputs from the host classifier alone are identified on the left column and outputs from the integrated classifier are identified on the right column. As shown in the Definite subjects 2102, two subjects were initially identified by the host classifier as not having an LRTI (less than 50% probability of having LRTI). By incorporating microbial data, however, the integrated classifier can identify that these two subjects have a significantly higher likelihood of having LRTI. For example, a presence of a particular virus and bacterial pathogens identified by metagenomics may have affected the increase of the probability values. For No Evidence subjects 2104, six subjects were initially identified by the host classifier as having an LRTI but were reclassified as not having any LRTI based on the results generated by the integrated classifier.

We note that at a much lower out-of-fold probability threshold of 15%, the integrated classifier's sensitivity for LRTI in the Definite group rose to >98%, suggesting a use-case as a rule-out test for LRTI.

### C. Determining a Classification of LRTI in Suspected and Indeterminate Cases

Finally, we trained the integrated classifier on all the Definite and No Evidence patients and then applied the trained integrated classifier to the Suspected and Indeterminate subjects, whose clinical diagnosis was less certain.

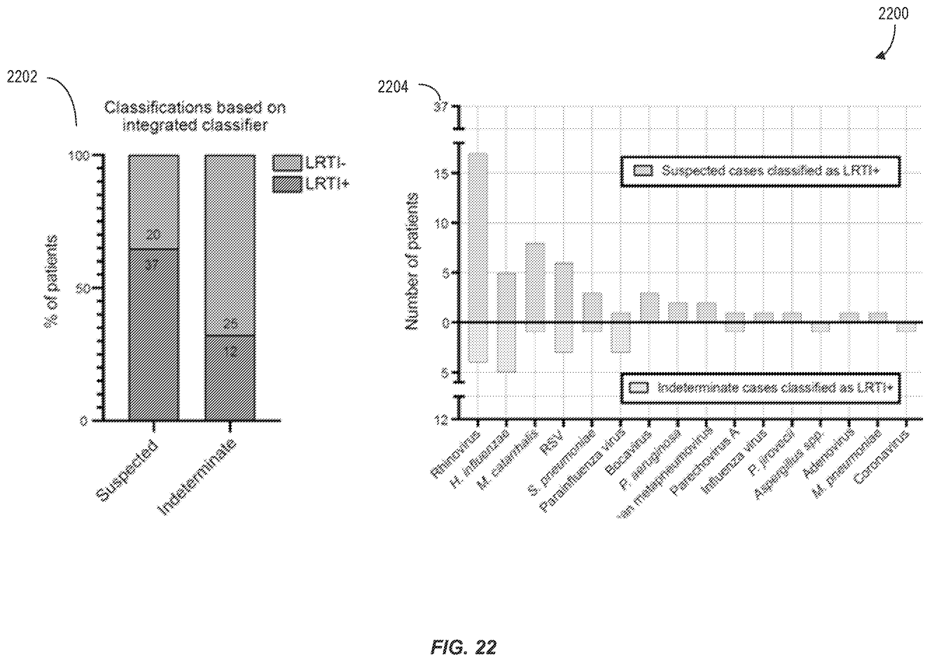

FIG. 22 shows evaluation results 2200 of the integrated classifier on subject suspected of LRTI, according to some embodiments. In FIG. 22, a bar plot 2202 shows a number and percentage of Suspected and Indeterminate patients that were classified as LRTI+ by the integrated classifier using a 50% probability threshold. The integrated classifier indicated 37/57 (65%) Suspected subjects as having LRTI and 12/37 (32%) Indeterminate subjects as having LRTI, consistent with the stronger clinical suspicion of LRTI in the former group. For the Suspected subjects predicted as having LRTI by the integrated classifier, the diversity model additionally called potential pathogens in 19/37 (51%). For comparison, the host classifier alone predicted infection in 40/57 (70%) of subjects with suspected LRTI but negative clinical testing, and the diversity model independently identified potential pathogens in 43/57 (75%) of subjects in the same group.

For the Indeterminate subjects, the host-only classifier predicted infection in 20/43 (47%) subjects, and the diversity model independently called potential pathogens in 19/43 (43%) subjects. The frequency of LRTI classification was higher in individual classifiers relative to that of the integrated classifier that identified 12/37 (32%) subjects of having LRTI.

In addition, graph 2204 of FIG. 22 identifies different species of viruses detected by mNGS and bacteria/fungi identified by the diversity model across the patients classified as LRTI+. Across all n=52 patients classified as LRTI+ in these two groups, likely pathogens (viral, bacterial, or fungal) were identified in 51 patients (98%). The detected pathogens included common (e.g., rhinovirus, H. influenzae), uncommon (e.g., bocavirus, parechovirus), and difficult to culture (e.g., Mycoplasma pneumoniae) microbes.

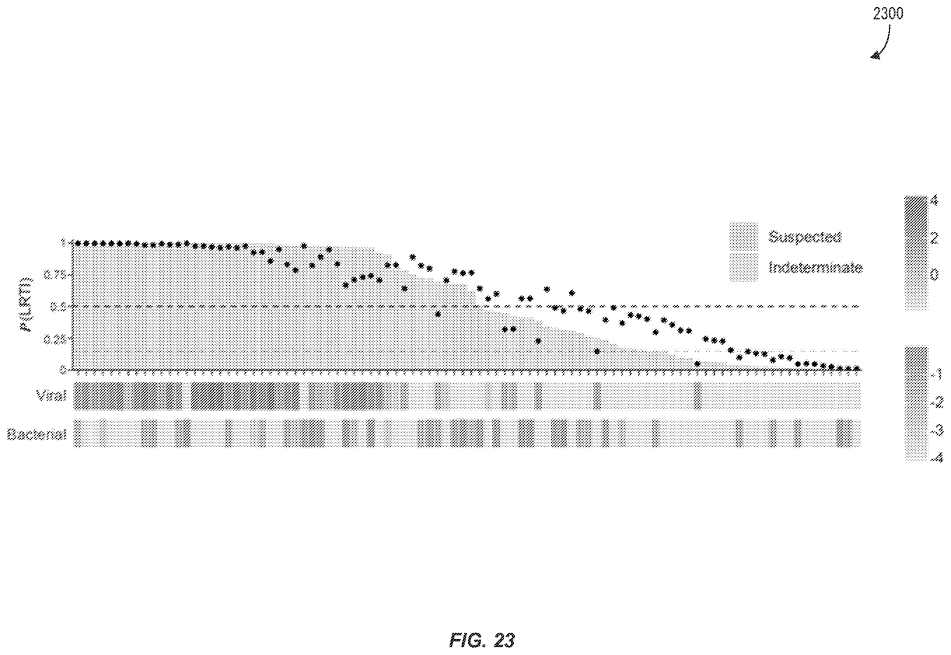

FIG. 23 also shows a visual summary 2300 incorporating all three inputs of the integrated classifier and its output LRTI probability for Suspected and Indeterminate cases. FIG. 23 thus provides of inputs (e.g., host probability) and output of the integrated classifier for all Suspected and Indeterminate patients. In particular, the top bars denote the integrated probability of LRTI and are colored by patient group. The black dots represent the input host LRTI probability, and the bottom vertical bars show the input log₁₀-transformed viral and bacterial scores. In addition, the dashed lines indicate the 50% probability of LRTI decision threshold and the 15% probability of LRTI ‘rule-out’ threshold.

### D. Method

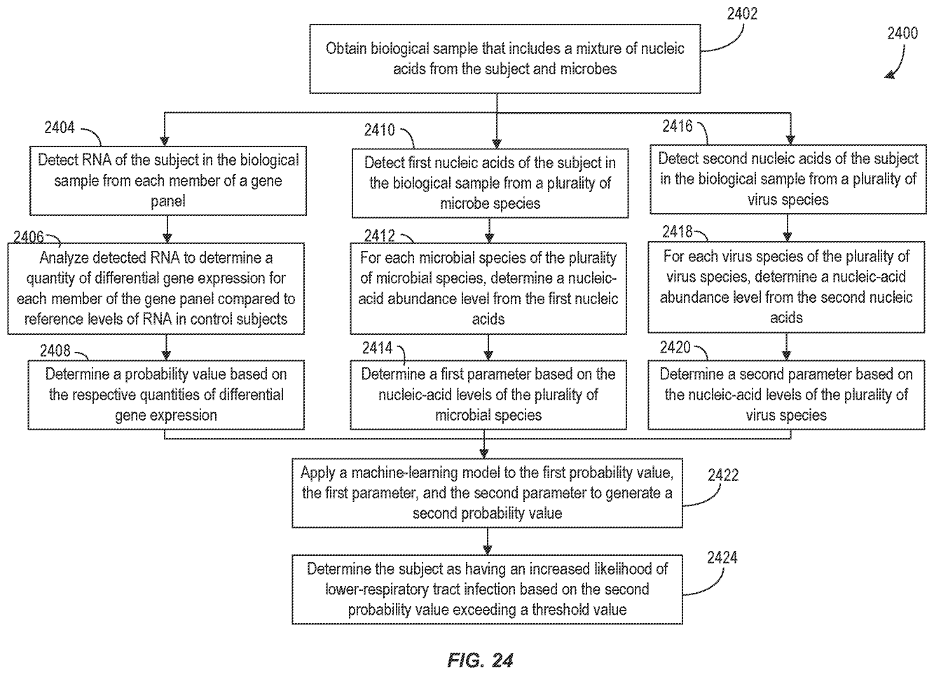

FIG. 24 is a flowchart for a method 2400 for using an integrated classifier to determine a likelihood of LRTI in a subject, according to some embodiments. At least a portion of the method may be performed by a computer system.

At block 2402, a biological sample of a subject is obtained. The biological sample can include a mixture of RNA from the subject and microbes. Exemplary biological samples are described herein and include those obtained, for example, by a nasal swab, nasopharyngeal swab, nasopharyngeal wash or aspirate, mid-turbinate nasal swab, oropharyngeal swab, buccal swab, a broncho-alveolar lavage, or an endotracheal aspirate. In some embodiments, the biological sample is serum, plasma, blood, or solid tissue. In some embodiments, a sample may be processed to provide or purify RNA of a particular nucleic acid molecule or fragment thereof.

At block 2404, RNA of the subject in the biological sample from each member of a gene panel is detected. In some instances, the gene panel can include at least two members selected from a group of genes consisting of GNLY, PSMB8, FFAR3, SLC38A2, ISG15, IRF1, KIAA1841, AC090425.2, AKR1C3, CXCL5, SESN1, PCOLCE2, RBP4, TAP1, EPSTI1, and FABP4. The steps for detecting RNA in the biological sample is further described in block 404 of FIG. 4.

At block 2406, the detected RNA is analyzed to determine a quantity of differential gene expression for each member of the gene panel compared to reference levels of RNA in control subjects. The quantity of the differential gene expression can include quantity and/or the frequency of RNA present in the biological sample as compared to reference levels in control subjects. The steps for determining the quantity of differential gene expression are further described in block 406 of FIG. 4.

At block 2408, a first probability value based on the respective quantities of differential gene expression is determined. The first probability value can correspond to an initial predicted likelihood of the subject of having an LRTI. A relationship between the respective quantities and the likelihood (probability) of having LRTI can be determined, e.g., using a proportion of samples having LRTI that have a given quantity of differential expression. In some instances, a machine learning model is used to provide the first probability value. The steps for determining the first probability value based on the respective quantities of differential gene expression are further described in block 408 of FIG. 4. In addition, the steps for determining the first probability value using the machine-learning model are further described in block 808 of FIG. 8

At block 2410, first nucleic acids of the subject in the biological sample are detected. In some instances, each nucleic acid is from a particular species of microbes of a plurality of species of microbes. In some instances, each nucleic acid is from a particular species of microbes of a plurality of species of microbes. To determine the nucleic acids from the species of microbes, nucleic acids from the subject can filtered out (e.g., sequences that align to a human reference genome). For example, the sequence reads of the biological sample can be aligned to the human reference genome. A subset of sequence reads aligning to the human reference genome can be filtered out. The remaining sequence reads can then be realigned to the one or more reference microbe genomes. A sequence read of the remaining sequence reads that aligns to a reference microbe genome of a particular microbe species can be determined the sequence read as being associated the particular microbe species. The steps for detecting the first nucleic acids are further described in block 1704 of FIG. 17.

At block 2412, for each microbial species of the plurality of microbial species, a nucleic-acid abundance level from the detected nucleic acids is determined. In some instances, abundance levels of microbial species are determined by determining the number of sequence reads (e.g., rpM) that are mapped to individual species of microbes. The steps for determining the abundance levels are further described in block 1706 of FIG. 17.

At block 2414, a first parameter is determined based on the nucleic-acid abundance levels of the plurality of microbial species. The first parameter can be indicative of an extent of microbial diversity in the biological sample. For example, the first parameter can correspond to a statistical value (e.g., a weighted sum) of abundance levels of a set of microbe species, in which each species the set of microbe species has an abundance level above a gap threshold. The steps for determining the gap threshold and the first parameter are further described in block 1708 of FIG. 17.

At block 2416, second nucleic acids in the biological sample are detected. In some instances, each of the second nucleic acids is from a particular virus species of a plurality of virus species. To determine the nucleic acids from the species of virus, nucleic acids from the subject can filtered out (e.g., sequences that align to a human reference genome). For example, the sequence reads of the biological sample can be aligned to the human reference genome. A subset of sequence reads aligning to the human reference genome can be filtered out. The remaining sequence reads can then be realigned to the one or more reference virus genomes. A sequence read of the remaining sequence reads that aligns to a reference virus genome of a particular virus species can be determined the sequence read as being associated the particular virus species.

At block 2418, for each virus species of the plurality of virus species, a nucleic-acid abundance level of the virus species from the second nucleic acids is determined. To determine the abundance level of the virus species, background filtering can be performed. For example, negative control samples consisting of only double-distilled water can also be processed with the biological sample. The negative control samples provide estimation of the number of background reads expected for each taxon, e.g., as described by Mick et al, Nature Communications 11:5854, 2020). The viruses with known ability to cause LRTI that were present at an abundance statistically exceeding their background distribution were considered probable pathogens.

At block 2420, a second parameter based on the nucleic-acid abundance levels of the plurality of virus species is determined. For example, the second parameter can correspond to a statistical value (e.g., a weighted sum) of abundance levels of a set of virus species, in which each species of the set of virus species has abundance levels statistically exceeding its corresponding background distribution. Accordingly, determining the second parameter can include aggregating the nucleic-acid abundance levels of the plurality of virus species. In some instances, the set of virus species corresponds to pathogen species known to cause LRTI.

At block 2422, a machine-learning model is applied to the first probability value, the first parameter, and the second parameter to generate a second probability value. The second probability value can correspond to a modified predicted likelihood of the subject of having an LRTI. The second probability value can be a different value from the first probability value, although the classification of LRTI of the subject may remain the same or change. The machine-learning model can be a logistic regression model. In some embodiments, each of the first probability value, the first parameter, and the second parameter is weighted with a predefined coefficient. The predefined coefficients can be the same or different. The probability score can be determined in various ways, e.g., by statistical or machine learning regression or classification such as, but not limited to, linear regression, including least squares regression, ridge or LASSO regression, elastic net regression, regularized Cox regression, logistic regression, orthogonal matching pursuit models, a Bayesian regression model, or deep learning methods, such as convolutional neural networks, recurrent neural networks and generative adversarial networks (see, e.g., LeCun et al., Nature 521: 436-444, 2015).

At block 2424, the subject is determined as having an increased likelihood of lower-respiratory tract infection based on the second probability score exceeding a threshold value. The threshold value can be determined in various ways, as described herein, e.g., it may correspond to 50% probability. The threshold value can be selected based on a desired accuracy, e.g., a trade off of sensitivity and specificity. In some embodiments, likelihood of LRTI is assigned based on a cutoff value using a reference scale, e.g., from 0 to 1.0. In some embodiments, a cutoff value of 0.5 or greater may be employed to define likelihood of LRTI. In some embodiments, LRTI likelihood may be further stratified, for example, likelihood of LRTI may be categorized as “high,” “intermediate,” or “low”, e.g., based on the highest tertile, intermediate tertile and bottom tertile.

### VI. Measuring Gene Expression Levels

Techniques and methods for measuring the expression levels of human genes and for are available in the art. For example, measuring the expression level of genes listed in Table 2 or Table 3 and the detection of genes of the gene panel may be accomplished by any suitable amplification method, such as polymerase chain reaction (PCR) methods and isothermal amplification methods (see section VI.A and VI.B below). Isothermal amplification methods that may be used to measure gene expression levels include, for example, loop-mediated isothermal amplification (LAMP). In some approaches, sequencing technologies may be used to quantify gene expression levels (e.g., metagenomic next generation sequencing; described in section VI.C, below). Other methods that may be used for measuring gene expression levels include but are not limited to hybridization capture methods, microarray analysis, Northern blot, serial analysis of gene expression (SAGE), and immunoassays. These methods are described, for example, in Sambrook and Russel (2001), Molecular Cloning: A Laboratory Manual, 3rd Edition, Cold Spring Harbor, N.Y.: Cold Spring Harbor Laboratory Press; Velculescu et al., 1995, Science 270:484-7; Serial Analysis of Gene Expression (SAGE): Methods and Protocols (Methods in Molecular Biology), Humana Press, 2008; herein incorporated by reference in their entireties.

### A. Methods Based on Polymerase Chain Reaction (PCR)

In some approaches, a polymerase chain reaction (PCR) may be used to measure the gene expression levels in subjects for determining a likelihood of LRTI. PCR-based methods that may be used include but are not limited to quantitative PCR (qPCR or real-time PCR), reverse transcriptase PCR (RT-PCR), and digital PCR. PCR methods are well known in the art, and are described, for example, in Innis et al., eds., PCR Protocols: A Guide To Methods And Applications, Academic Press Inc., San Diego, Calif. (1990); see Sambrook and Russel (2001), Molecular Cloning: A Laboratory Manual, 3rd Edition, Cold Spring Harbor, N.Y.: Cold Spring Harbor Laboratory Press; Chapter 8: In vitro Amplification of DNA by the Polymerase Chain Reaction; PCR Technology: Principles and Applications for DNA Amplification (ed. H. A. Erlich, Freeman Press, N.Y., N.Y., 1992, herein incorporated by reference in their entirety.

In some approaches, quantitative reverse transcriptase PCR (qRT-PCR) may be used. The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMY-RT) and Moloney murine leukemia virus reverse transcriptase (MLVRT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction. Although the PCR step can use a variety of thermostable DNAdependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, may be designed to detect nucleotide sequence located between the two PCR primers. The probe is non-extendible by Taq DNA polymerase enzyme and may be labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data. See, e.g. Real-Time PCR: Current Technology and Applications, Logan, Edwards, and Saunders eds., Caister Academic Press, 2009; Joyce (2002), “Quantitative RT-PCR. A review of current methodologies,” Methods Mol. Biol. 193. pp. 83-92; Bustin et al. (2005), “Quantitative real-time RT-PCR—a perspective,” J. Mol. Endocrinol. 34 (3): 597-601; Bustin (2000), “Absolute quantification of mRNA using real-time reverse transcription polymerase chain reaction assays,” J. Mol. Endocrinol. 25 (2): 169-93; Deepak et al. (2007), “Real-Time PCR: Revolutionizing Detection and Expression Analysis of Genes”. Curr. Genomics. 8 (4): 234-51; Gause et al. (1994). “The use of the PCR to quantitate gene expression”. PCR Methods Appl. 3 (6): S123-35.

Accordingly, in some approaches measuring the expression level of the one or more genes shown in Table 2 or Table 3 comprises performing PCR (e.g., qRT-PCR). The PCR may be performed by using at least one set of oligonucleotide primers comprising a forward primer and a reverse primer capable of amplifying a polynucleotide sequence of the gene (such as IFI6). Methods for the design and/or production of nucleotide primers are generally known in the art, and are described in e.g., Sambrook et al. (2001) Molecular Cloning: A Laboratory Manual (3rd ed., Cold Spring Harbor Laboratory Press, Plainview, N.Y.); Ausubel F. M. et al. (Eds) Current Protocols in Molecular Biology (2007), John Wiley and Sons, Inc; Molecular Cloning: A Laboratory Manual, 4th ed., Green and Sambrook, 2012). Nucleotide primers and probes may be prepared, for example, by chemical synthesis techniques for example, the phosphodiester and phosphotriester methods (see for example Narang S. A. et al. (1979) Meth. Enzymol. 68:90; Brown, E. L. (1979) et al. Meth. Enzymol. 68:109; and U.S. Pat. No. 4,356,270), the diethylphosphoramidite method (see Beaucage S. L et al. (1981) Tetrahedron Letters, 22:1859-1862). Oligonucleotide primers are typically being between 5-80 nucleotides in length, e.g., between 10-50 nucleotides in length, or between 15-30 nucleotides in length. Any appropriate length of sequence may be used such as 10, 11, 12, 13, 14, 15, 16, 17, 18, 19, 20, 21, 22, 23, 24, 25, 26, 27, 28, 29, or 30 nucleotides or more.

### B. Isothermal Amplification Methods

In some embodiments, isothermal amplification methods can be used to measure the expression level of the genes. A number of isothermal amplification methods are known in the art and have been discussed, e.g., in Zhao et al. (2015), “Isothermal amplification of nucleic acids,” Chem. Rev., 115 (22), 12491-12545; Niemz et al. (2011), “Point-of-care nucleic acid testing for infectious diseases,” Trends Biotechnol.; 29:240-250; Yan et al. 92014), “Isothermal amplified detection of DNA and RNA,” Mol. Biosyst. 10, 970-1003. Any suitable isothermal amplification method may be used. In some approaches, loop-mediated isothermal amplification (LAMP) may be used. For example, LAMP may be particularly suitable for point of care (POC) settings as the method typically operates at 60-65° C. to achieve exponential amplification of nucleic acid targets without requiring temperature cycling. LAMP methods are known in the art and described, e.g., in U.S. Pat. No. 6,410,278; Notomi et al. (2000), “Loop-mediated isothermal amplification of DNA,” Nucleic Acids Res.; 28:E63; Nagamine et al. (2002), “Accelerated reaction by loop-mediated isothermal amplification using loop primers,” Mol. Cell. Probes. 16 (3): 223-9; Tomita et al. (2008), “Loop-mediated isothermal amplification (LAMP) of gene sequences and simple visual detection of products,” Nat. Protoc. 3, 877-82; Fu et al. (2011), “Applications of loop-mediated isothermal DNA amplification,” Appl. Biochem. Biotechnol. 163, 845-50. LAMP is a one-step amplification system using auto-cycling strand displacement DNA synthesis. The target sequence is amplified at a constant temperature of 60-65° C. using either two or three sets of primers and a polymerase with high strand displacement activity in addition to a replication activity. Typically, 4 different primers are used to amplify 6 distinct regions on the target gene, which increases specificity. An additional pair of “loop primers” can further accelerate the reaction. The amplification product can be detected via photometry, measuring the turbidity caused by magnesium pyrophosphate precipitate in solution as a byproduct of amplification.

Other isothermal amplification methods that may be used include but are not limited to transcription-mediated amplification (TMA) Nucleic Acid Sequence Based Amplification (NASBA), Multiple Displacement Amplification (MDA), Rolling Circle Amplification (RCA), Helicase Dependent Amplification (HDA), Strand Displacement Amplification (SDA), Nicking Enzyme Amplification Reaction (NEAR), Ramification Amplification Method (RAM), and Recombinase Polymerase Amplification (RPA). In some approaches, TMA is used to measure the expression level of the genes.

### C. Sequencing Technologies

The gene expression levels may be measured using sequencing technologies, such as next generation sequencing platforms (e.g., RNA-Seq). RNA-SEQ uses next-generation sequencing (NGS) for the detection and quantification of RNA in a biological sample at a given moment in time. An RNA library is prepared, transcribed, fragmented, sequenced, reassembled and the sequence or sequences of interest quantified. NGS methods are well known in the art and described e.g., in Mortazavi et al., Nat. Methods 5: 621-628, 2008; Karl et al. (2009), “Next-Generation Sequencing: From Basic Research to Diagnostics,” Clinical Chemistry. 55 (4): 641-658; Wang et al. (2009), “RNA-Seq: a revolutionary tool for transcriptomics,” Nature Reviews. Genetics. 10 (1): 57-63; Kukurba and Montgomery (2015), “RNA Sequencing and Analysis”, Cold Spring Harbor Protocols., (11): 951-69. In some approaches, whole transcriptome shotgun sequencing may be used to measure gene expression levels. In some approaches, metagenomics NGS (mNGS) may be used to measure gene expression levels. See e.g., Chiu and Miller (2019), “Clinical metagenomics,” Nature Reviews Genetics, 20 (6): 341-355; Maljkovic et al. (2019), “Next Generation Sequencing and Bioinformatics Methodologies for Infectious Disease Research and Public Health: Approaches, Applications, and Considerations for Development of Laboratory Capacity,” The Journal of Infectious Diseases: jiz286; Wilson et al. (2019), “Clinical metagenomic sequencing for diagnosis of meningitis and encephalitis,” N. Engl. J. Med. 380, 2327-2340. Exemplary sequencing platforms suitable for use according to the methods include, e.g., ILLUMINA® sequencing (e.g., HiSeq, MiSeq), SOLID® sequencing, ION TORRENT® sequencing, and SMRT® sequencing and those commercialized by Roche 454 Life Sciences (GS systems).

### VI. Treatments

LRTI is generally treated with antimicrobials targeted to the underlying pathogen(s) causing the disease. Antimicrobials can include antibacterial antibiotics such as cefazolin, ceftriaxone, cefepime, meropenem, vancomycin, azithromycin, ciprofloxacin, levofloxacin; or antiviral agents such as oseltamivir (influenza), remdesivir, paxlovid (SARS-CoV-2), or palivizumab (RSV). The methods described herein enable more accurate identification of LRTI, and precision detection of the responsible pathogen(s) enabling targeted, as opposed to the standard approach of blind, empiric broad-spectrum that may or may not have efficacy against the causative microbe(s). Other types of antibiotics can include, for example, cefotaxime, ceftazidime, cefuroxime, nafcillin, oxacillin, ampicillin, ticarcillin, ticarcillin/clavulinic acid, ampicillin/sulbactam (Unasyn), trimethoprim-sulfamethoxazole, clindamycin, synercid, amoxicillin, amoxicillin/clavulinic acid, cefuroxime, trimethoprim/sulfamethoxazole, clindamycin, dicloxacillin, cefixime, cefpodoxime, loracarbef, cefadroxil, cefabutin, cefdinir, and cephradine.

### VII. Computer System

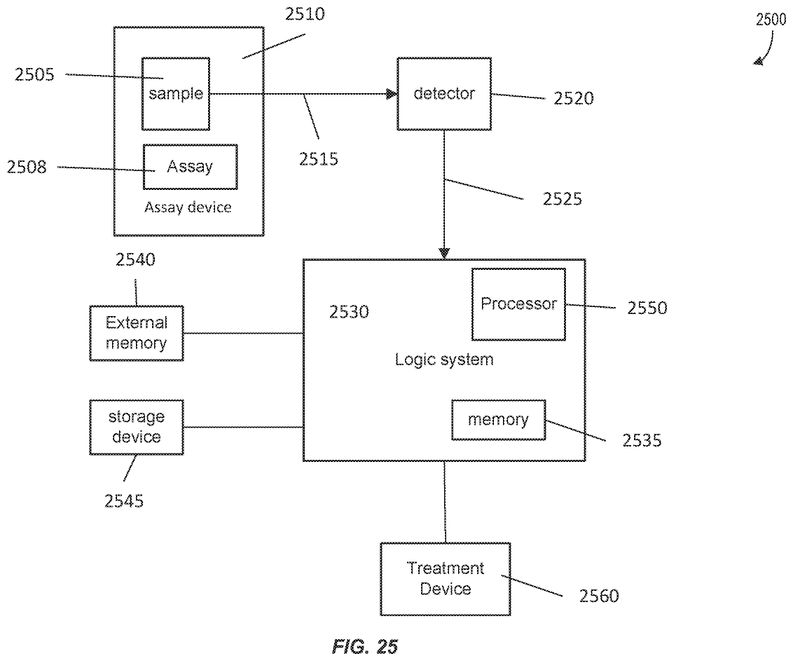

FIG. 25 illustrates a measurement system 2500 according to an embodiment of the present disclosure. The system as shown includes a sample 2505, such as DNA or RNA molecules within an assay device 2510, where an assay 2508 can be performed on sample 2505. For example, sample 2505 can be contacted with reagents of assay 2508 to provide a signal of a physical characteristic 2515. An example of an assay device can be a flow cell that includes probes and/or primers of an assay or a tube through which a droplet moves (with the droplet including the assay). Physical characteristic 2515 (e.g., a fluorescence intensity, a voltage, or a current), from the sample is detected by detector 2520. Detector 2520 can take a measurement at intervals (e.g., periodic intervals) to obtain data points that make up a data signal. In one embodiment, an analog-to-digital converter converts an analog signal from the detector into digital form at a plurality of times. Assay device 2510 and detector 2520 can form an assay system, e.g., a sequencing system that performs sequencing according to embodiments described herein. A data signal 2525 is sent from detector 2520 to logic system 2530. As an example, data signal 2525 can be used to determine sequences and/or locations in a reference genome of DNA molecules. Data signal 2525 can include various measurements made at a same time, e.g., different colors of fluorescent dyes or different electrical signals for different molecule of sample 2505, and thus data signal 2525 can correspond to multiple signals. Data signal 2525 may be stored in a local memory 2535, an external memory 2540, or a storage device 2545.

Logic system 2530 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 2530 and the other components may be part of a stand-alone or network connected computer system, or they may be directly attached to or incorporated in a device (e.g., a sequencing device) that includes detector 2520 and/or assay device 2510. Logic system 2530 may also include software that executes in a processor 2550. Logic system 2530 may include a computer readable medium storing instructions for controlling measurement system 2500 to perform any of the methods described herein. For example, logic system 2530 can provide commands to a system that includes assay device 2510 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

System 2500 may also include a treatment device 2560, which can provide a treatment to the subject. Treatment device 2560 can determine a treatment and/or be used to perform a treatment. Examples of such treatment can include surgery, radiation therapy, chemotherapy, immunotherapy, targeted therapy, hormone therapy, and stem cell transplant. Logic system 2530 may be connected to treatment device 2560, e.g., to provide results of a method described herein. The treatment device may receive inputs from other devices, such as an imaging device and user inputs (e.g., to control the treatment, such as controls over a robotic system).

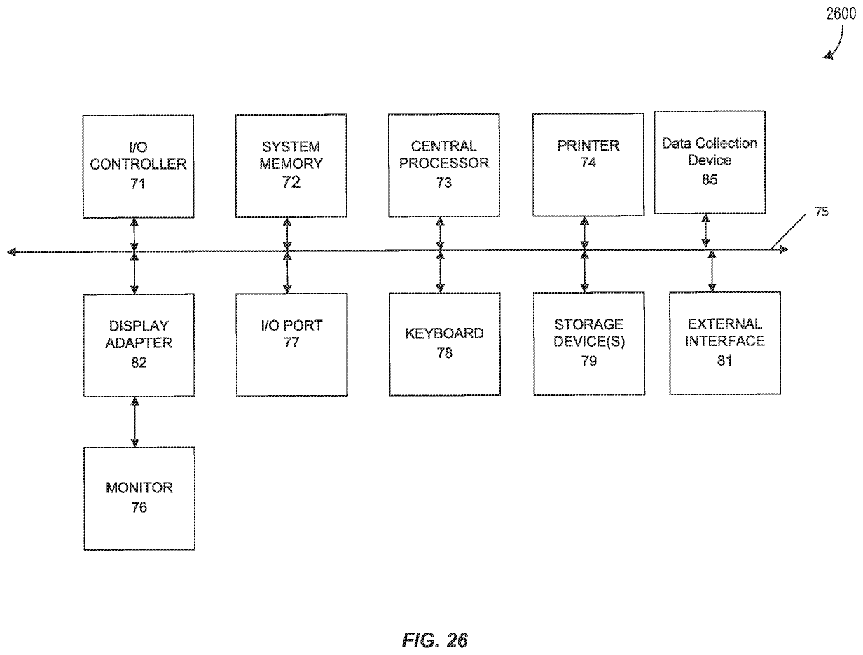

Any of the computer systems mentioned herein may utilize any suitable number of subsystems. Examples of such subsystems are shown in FIG. 26 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 26 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

1. A method of analyzing a biological sample to determine a likelihood of lower-respiratory tract infection in a subject, the biological sample including a mixture of RNA from the subject and microbes, the method comprising:
detecting RNA of the subject in the biological sample from each member of a gene panel, wherein the gene panel comprises at least two members selected from a group consisting of GNLY, PSMB8, FFAR3, SLC38A2, ISG15, IRF1, KIAA1841, AC090425.2, AKR1C3, CXCL5, SESN1, PCOLCE2, RBP4, TAP1, EPSTI1, and FABP4;
determining, from the detected RNA, a quantity of differential gene expression for each member of the gene panel compared to reference levels of RNA in control subjects;
determining a probability value based on the respective quantities of differential gene expression; and
determining the subject as having an increased likelihood of lower-respiratory tract infection based on the probability value exceeding a threshold value.

2. The method of claim 1, wherein determining the probability value includes applying a machine-learning model to the respective quantities of differential gene expression to generate the probability value.

3. The method of claim 2, wherein the machine-learning model is a random forest classifier.

4. The method of claim 1, wherein the threshold value corresponds to 50% probability.

5. The method of claim 1, wherein determining the probability value uses a weighted sum of the respective quantities of differential gene expression.

6. The method of claim 1, wherein the gene panel comprises at least two members selected from the group consisting of GNLY, PSMB8, FFAR3, SLC38A2, ISG15, IFR1, RBP4, and FABP4.

7. The method of claim 1, wherein the gene panel comprises at least two members selected from the group consisting of TAP1, FABP4, RBP4, EPSTI1, and FFAR3.

8. The method of claim 1, wherein the gene panel comprises at least two members selected from the group consisting of TAP1, FABP4, and RBP4.

9. A method of analyzing a biological sample to determine a likelihood of lower-respiratory tract infection in a subject, the biological sample including a mixture of nucleic acids from the subject and microbes, the method comprising:
detecting nucleic acids in the biological sample, wherein each nucleic acid is from a particular species of microbes of a plurality of microbial species;
determining, for each microbial species of the plurality of microbial species, a nucleic-acid abundance level from the detected nucleic acids;
determining a parameter based on the nucleic-acid abundance levels of the plurality of microbial species, wherein the parameter is indicative of an extent of microbial diversity in the biological sample; and
determining the subject as having an increased likelihood of lower-respiratory tract infection based on the parameter indicating the extent of microbial diversity is below a threshold.

10. The method of claim 9, wherein the threshold is determined based on one or more reference subjects having a known classification of whether a lower-respiratory tract infection exists.

11. The method of claim 9, wherein the parameter is a diversity index, wherein the diversity index is generated at least by:
normalizing, for each microbial species of the plurality of microbial species, the nucleic-acid abundance level of the microbial species; and
determining a negative sum of the normalized nucleic-acid abundance levels.

12. The method of claim 9, wherein determining the parameter based on the nucleic-acid abundance levels includes:
determining a gap threshold, wherein the gap threshold is the nucleic-acid abundance level at which a greatest difference in the nucleic-acid abundance level occurs between the plurality of microbial species.

13. The method of claim 12, wherein determining the likelihood of lower-respiratory tract infection in the subject includes:
identifying one or more microbial species from the plurality of microbial species, wherein each of the one or more microbial species has the nucleic-acid abundance level that is above the gap threshold; and
determining the subject as having the increased likelihood of lower-respiratory tract infection based on the one or more microbial species.

14. The method of claim 9, wherein detecting the RNA of the subject in the biological sample from each member of the gene panel includes amplifying RNA molecules from the gene panel.

15. The method of claim 9, wherein detecting the RNA of the subject in the biological sample from each member of the gene panel includes performing sequencing of RNA molecules.

16. A method of analyzing a biological sample to determine a likelihood of lower-respiratory tract infection in a subject, the biological sample including a mixture of nucleic acids from the subject and microbes, the nucleic acids including RNA, the method comprising:
detecting RNA of the subject in the biological sample from each member of a gene panel, wherein the gene panel comprises at least two members selected from a group consisting of GNLY, PSMB8, FFAR3, SLC38A2, ISG15, IRF1, KIAA1841, AC090425.2, AKR1C3, CXCL5, SESN1, PCOLCE2, RBP4, and FABP4;
determining, from the detected RNA, a quantity of differential gene expression for each member of the gene panel compared to reference levels of RNA in control subjects;
determining a first probability value based on the respective quantities of differential gene expression;
detecting first nucleic acids in the biological sample, wherein each of the first nucleic acids is from a microbial species of a plurality of microbial species;
determining, for each microbial species of the plurality of microbial species, a nucleic-acid abundance level from the first nucleic acids;
determining a gap threshold, wherein the gap threshold is the nucleic-acid abundance level at which a greatest difference in nucleic-acid abundance level occurs between the plurality of microbial species;
identifying one or more microbial species from the plurality of microbial species, wherein each of the one or more microbial species has the nucleic-acid abundance level that is above the gap threshold;
determining a first parameter based on nucleic-acid abundance levels corresponding to the one or more microbial species;
detecting second nucleic acids in the biological sample, wherein each of the second nucleic acids is from a particular virus species of a plurality of virus species;
determining, for each virus species of the plurality of virus species, a nucleic-acid abundance level of the virus species from the second nucleic acids;
determining a second parameter based on the nucleic-acid abundance levels of the plurality of virus species;
applying a machine-learning model to the first probability value, the first parameter, and the second parameter to generate a second probability value; and
determining the subject as having an increased likelihood of lower-respiratory tract infection based on the second probability value exceeding a threshold value.

17. The method of claim 16, wherein determining the first probability value includes applying another machine-learning model to the respective quantities of differential gene expression to generate the first probability value.

18. The method of claim 16, wherein the first parameter is a diversity index, wherein the diversity index is generated at least by:
normalizing, for each microbial species of the plurality of microbial species, the nucleic-acid abundance level of the microbial species; and
determining a negative sum of the normalized nucleic-acid abundance levels.

19. The method of claim 16, wherein determining the second parameter include aggregating the nucleic-acid abundance levels of the plurality of virus species.

20. The method of claim 16, wherein the first nucleic acids and the second nucleic acids are the same nucleic acids.

21. The method of claim 1, wherein detecting the RNA of the subject in the biological sample from each member of the gene panel includes amplifying RNA molecules from the gene panel.

22. The method of claim 1, wherein detecting the RNA of the subject in the biological sample from each member of the gene panel includes performing sequencing of RNA molecules.

23-27. (canceled)
